(19) United States
(12) Patent Application Publication
(10) Pub. No.: US 2023/0201203 A1
(43) Pub. Date: Jun. 29, 2023

(54) METHODS OF TREATMENT USING ANTIFOLATES AND PHARMACEUTICAL FORMULATIONS COMPRISING ANTIFOLATES

(71) Applicant: EMPHASCIENCE, INC., Pittsford, NJ (US)

(72) Inventor: Rebanta BANDYOPADHYAY, Webster, NY (US)

(21) Appl. No.: 17/995,418

(22) PCT Filed: Apr. 9, 2021
(86) PCT No.: PCT/US2021/070372

§ 371 Date: Oct. 4, 2022

Related U.S. Application Data
(60) Provisional application No. 63/007,471, filed on Apr. 9, 2020.

(51) Int. Cl. A61K 31/519; A61K 31/198; A61K 9/00

(52) U.S. Cl. CPC A61K 31/519; A61K 31/198; A61K 9/0019

(57) ABSTRACT

Herein is disclosed, a method of treating a viral pulmonary infection, the method comprising administering to a subject in need thereof a therapeutically effective amount of an antifolate.

### TECHNICAL FIELD

The present disclosure relates to methods of treating viral pulmonary infections using antifolates. The present disclosure also relates to novel pharmaceutical formulations comprising antifolates.

### BACKGROUND

A novel viral pulmonary infection COVID-19 surfaced in 2019. Previous novel viral pulmonary infections included SARS, H1N1 and MERS which surfaced in 2002, 2009 and 2012 respectively. As of 31st March 2020, 219 countries and territories have reported about 128.3 million Covid-19 cases and 2.8 million deaths due to COVID-19 since the first cases were recorded in China in December 2019. While the infection remains at large, there are no clinically approved drugs to treat all forms and stages of COVID-19. The rapid emergence and spread of such viral infections, including the appearance of more virulent mutations, allow little time for the development and testing of vaccines. In the absence of clinically effective and safe vaccines or widespread immunization against all mutations and variants, controlling the virus and taking care of the sick become almost impossible unless there are effective therapeutics to address the gaps left behind by unreliable or ineffective vaccination.

There is therefore a rapid and continuing need to develop new methods of treating viral pulmonary infections, such as COVID-19.

Antiviral drug discovery has previously mostly focused on viral targets. Indeed, 88% of all approved antiviral drugs to date are designed to directly target the virus. However, drugs that act against viral targets may have a narrow spectrum of activity and may only be specific to one type of virus. Such drugs may therefore be ineffective against a broad class of viral pulmonary infections. Drugs that act against the viral target may also lose their efficacy as a result of viral mutation leading to the development of resistance.

### SUMMARY

In a first aspect, there is provided a method of treating a viral pulmonary infection, the method comprising administering to a subject in need thereof a therapeutically effective amount of an antifolate. In some embodiments, the method of treatment may comprise administration of the antifolate by injection, for example, subcutaneous injection, intravenous injection, intramuscular injection, or intradermal injection. In some embodiments, the preferred method and route of administration is bolus intravenous injection to maximize the plasma level of the antifolate for the lowest possible dose and to maximize the time over which the plasma concentration is above the minimum effective concentration against the viral infection. In some embodiments, the antifolate is administered by intravenous bolus injection in multiple doses. In some embodiments such treatment using the antifolate can be carried out without the need for supplementing such therapy with pre-administration of vitamin B12 (cyanocobalamin) and/or folic acid, or coadministration of corticosteroids, such as, dexamethasone. In some embodiments, the viral pulmonary infection is a coronavirus infection, for example, SARS-CoV-2. In some embodiments, the antifolate is pemetrexed and/or salts thereof. In some embodiments, the antifolate is provided as a pharmaceutical formulation as described herein. In some embodiments, the antifolate dosage is from 0.01 mg/m² to about 700 mg/m² of body surface area, optionally from about 0.1 mg/m² to about 150 mg/m² of body surface area, further optionally from about 25 mg/m² to about 100 mg/m² of body surface area and further optionally from about 35 mg/m² to about 75 mg/m² of body surface area. In some embodiments, the amount of antifolate administered to the subject is from about 1 to about 200 mg, or from about 25 mg to about 175 mg, or optionally from about 35 mg to about 175 mg, or about 50 mg to about 150 mg. In some embodiments, one or more of the aforementioned embodiments are combined.

In a second aspect, there is provided a pharmaceutical formulation comprising an antifolate and/or salts thereof, sodium sulfite, and cysteine hydrochloride. The pharmaceutical formulation of the second aspect may be used to administer a therapeutically effective amount of antifolate in the method of treatment of the first aspect. In some embodiments, the antifolate is pemetrexed or salts thereof. In some embodiments, the osmolality of the pharmaceutical formulation is from about 250 mOsm to about 400 mOsm. In some embodiments, the pharmaceutical formulation comprises a tonicity adjuster, wherein the tonicity adjuster may be selected from NaCl, KCl, glucose, dextrose, mannitol or glycerin. In some embodiments, the weight ratio of the antifolate to cysteine hydrochloride is from about 12:1 to about 55:1, or about 13:1 to about 25:1, or about 14:1 to about 18:1. In some embodiments, the weight ratio of the antifolate to sodium sulfite is from about 2.5:1 to about 22:1, or from about 1:1.5 to about 1:2.5, or from about 4:1 to about 9:1. In some embodiments, the weight ratio of cysteine hydrochloride to sodium sulfite is from about is from about 1:1.5 to about 1:4.5, or from about 1:2.8 to about 1:3.1. In some embodiments, the antifolate is present in the aqueous solution in an amount of from about 2% w/w to about 10% w/w based on the total amount of aqueous solution, or about 2% w/w to about 8% w/w based on the total amount of aqueous solution, or from about 2.19 w/w to about 2.5% w/w or from about 4.0 to 5.5% w/w based on the total weight of the aqueous solution. In some embodiments, the cysteine hydrochloride monohydrate is present in the aqueous solution in an amount of from about 0.04% w/w to about 0.2% w/w based on the total amount of aqueous solution, or from 0.1% w/w to 0.2% w/w based on the total amount of aqueous solution. In some embodiments, the sodium sulfite is present in an amount of about 0.1% w/w to about 0.95% w/w based on the total amount of aqueous solution, or from 0.5% w/w to about 0.8% w/w based on the total amount of aqueous solution. In some embodiments, the aqueous solution comprises about 2% w/w to about 10% w/w of antifolate, 0.04% w/w to about 0.2% w/w cysteine hydrochloride monohydrate and 0.1% w/w to about 0.95% w/w sodium sulfite based on the total amount of aqueous solution. In some embodiments, the aqueous solution comprises 94% w/w to 97.9% w/w water based on the total amount of the aqueous solution. In some embodiments, the aqueous solution comprises NaOH in an amount from about 0.4% w/w to about 0.9% w/w based on the total amount of the aqueous solution. In some embodiments, the antifolate is present in the pharmaceutical formulation at a concentration from about 0.5 mg/mL to about 100 mg/mL, or from about 15 mg/mL to about 80 mg/mL, or from about 25 mg/mL to about 75 mg/mL or from about 40 mg/mL to about 55 mg/mL, or from about 60 mg/mL to about 80 mg/mL. In some embodiments, one or more of the aforementioned embodiments are combined.

In a third aspect, there is provided a method of treating lung cancer, the method comprising administering to a subject in need thereof the pharmaceutical formulation of the second aspect. In an embodiment, the antifolate is pemetrexed or salts thereof. While antifolates used in the treatment of cancers are known, there has been no such treatment with the novel pharmaceutical formulation described herein. The novel pharmaceutical formulations can be suitable for targeted delivery to the lung. In some embodiments, the method of treatment comprises parenteral administration. In some embodiments, the method of treatment comprises administration by intravenous infusion. In some embodiments, the method of treatment comprises administration by injection, for example, for example, subcutaneous injection, bolus intravenous injection, intramuscular injection, or intradermal injection. In an embodiment, the lung cancer is non-small cell lung cancer. One or more of these embodiments can be readily combined.

In a fourth aspect, there is provided a method of treating mesothelioma and/or other forms of similar malignant disease, the method comprising administering to a subject in need thereof the pharmaceutical formulation of the second aspect. In an embodiment, the antifolate is pemetrexed or salts thereof. In some embodiments, the method of treatment comprises parenteral administration. In some embodiments, the method of treatment comprises administration by intravenous infusion. In some embodiments, the method of treatment comprises administration by injection, for example, for example, subcutaneous injection, bolus intravenous injection, intramuscular injection, or intradermal injection. One or more of these embodiments can be readily combined.

In a fifth aspect, there is provided a method of treating an autoimmune disease, the method comprising administering to a subject in need thereof the pharmaceutical formulation of the second aspect. In an embodiment, the antifolate is pemetrexed or salts thereof. In some embodiments, the method of treatment comprises parenteral administration. In some embodiments, the method of treatment comprises administration by intravenous infusion. In some embodiments, the method of treatment comprises administration by injection, for example, for example, subcutaneous injection, bolus intravenous injection, intramuscular injection, or intradermal injection. One or more of these embodiments can be readily combined.

The first aspect of the present disclosure describes a novel method of treating viral pulmonary infections using antifolates. While such drugs have been used in the treatment of cancer, there has been no previous use against viral pulmonary infections. Without wishing to be bound by theory, it is thought that the method of treatment works by inhibiting the host cell biosynthetic pathway required by viruses to replicate. Folic acid and folates, and the folic acid-dependent pathways are essential for the synthesis of purine and pyrimidine nucleotides that are eventually incorporated into DNA or RNA. Viruses lack their own replication mechanism but utilize the folic acid pathway of host cells that they infect for replication of their DNA or RNA and for multiplication into new virions. The inhibition of the folate-dependent pathways is therefore considered to be a viable strategy for halting viral replication and treating viral infection.

The present disclosure also details novel pharmaceutical formulations comprising antifolates. The novel pharmaceutical formulations according to the present disclosure have in many cases demonstrated good stability and prolonged shelf-life as compared to previous pharmaceutical formulations that comprise antifolates. The novel pharmaceutical formulation is suitable for injection, as well as, targeted administration to the lung, and may be used in the treatment in viral pulmonary infections, mesothelioma and lung cancer (e.g. non-small cell lung cancer). Dosage of antifolate for viral pulmonary infections may be less than what is required for cancer treatment.

Certain embodiments of the present invention including the method of treatment and/or pharmaceutical formulations described herein may provide one or more of the following advantages:
slowing down or halting viral replication of viral pulmonary infections;
reducing in the likelihood of loss of therapeutic effectiveness due to development of viral resistance from mutation, as compared to drugs that are designed to act against virus-specific targets;
ability to administer multiple doses in very low increments and multiple times over shorter time intervals to maintain the plasma concentration just above effective anti-viral levels for longer period of time without achieving unnecessarily high plasma concentrations with an excessively high single dose that would otherwise rapidly fall below the effective antiviral concentration.
maximizing therapeutic effectiveness without compromising the safety of the patient;
achieving effective immediate treatment without the need for supplementation of the patient with dietary folates or folic acid, and vitamin B₁₂ and/or corticosteroids (sometimes several days prior to the initiation of therapy).
achieving efficacy against different types or strains of virus that cause viral pulmonary infections;
reducing the acute manifestation of the immunogenic and/or inflammatory response and sepsis from the so-called ‘cytokine storm’;
maintaining the effective antiviral concentration (EC₉₀) for long enough to effectively inhibit the replication of the infecting virus specifically at the lung and/or other sites of infection. This may be achieved, for example, by direct intravenous injection as a bolus dose, inhalation, or intratracheal instillation;
delivering the active agent directly to the local tissue-site or target for treatment;
delivering the active agent directly to the lung in a targeted manner;
achieving shorter administration times. This can be achieved using bolus intravenous injection without further dilution as opposed to e.g. intravenous infusion;
achieving low dose-volume formulations, e.g. by in some instances using a high concentration of formulation;
increasing cellular uptake in affected cells;
improving permeability and access to infected or target cells across mucous barriers by using pharmaceutical excipients such as mucolytic agents;
retaining the active agent at the site of administration for a longer period of time by constricting capillary blood flow and reducing systemic exposure. This may be achieved by added adrenergic agents at very low parts per million (ppm) levels;
obtaining flexible formulation that can be ready-to-administer or may easily be turned into a ready-to-administer solution by a single-step dilution;
improving stability for the antifolate against oxidation and hydrolysis or solvolysis;
stabilizing the formulation against precipitation at ambient or at sub-ambient (refrigerated) temperatures of storage over time as compared to other pharmaceutical formulations comprising antifolates;
ability to steam sterilize at 121° C., or similar temperatures ranging from 90 to 130° C., following compounding to ensure sterility, which may not be achieved with other antifolate injection formulations;
improving the therapeutic effect in patients with lung cancer. This may result from quicker and targeted delivery of the pharmaceutical formulation to the lung, for example, by parenteral administration as a bolus injection or a faster infusion using reduced dose-volume, or by inhalation, or by intratracheal instillation;
Improving the therapeutic effect in patients suffering from viral infections, such as COVID-19, in whom therapeutic intervention is delayed and the disease has progressed to more life threatening, peak manifestations of such viral infection.

The details, examples and preferences provided in relation to any one or more of the stated aspects of the present invention will be further described herein and apply equally to all aspects of the present invention. Any combination of the embodiments and preferences described herein in all possible variations thereof is encompassed by the present invention unless otherwise indicated herein, or otherwise clearly contradicted by context.

### BRIEF DESCRIPTION OF FIGURES

FIG. 1 shows A) the simulated plasma profile from 4 doses of IV bolus injections of pemetrexed administered 3 hours apart starting with a dose of 150 mg, followed by 3×75 mg doses and B) the simulated plasma profile from one 1000 mg dose (either bolus) or 10 min IV infusion as currently used in cancer chemotherapy.

### DETAILED DESCRIPTION

When ranges are used herein, all combinations and sub-combinations of ranges and specific embodiments therein are intended to be included. The term “about” when referring to a number or a numerical range means that the number or numerical range referred to is an approximation within experimental variability (or within statistical experimental error), and thus the number or numerical range may vary from, for example, between 1% and 15% of the stated number or numerical range.

The term “comprising” (and related terms such as “comprise” or “comprises” or “having” or “including”) includes those embodiments, for example, an embodiment of any composition of matter, composition, method, or process, or the like, that “consist of” or “consist essentially of” the described features.

Abbreviations used herein have their conventional meaning within the chemical and biological arts, unless otherwise indicated.

The term “effective amount” or “therapeutically effective amount” refers to the amount of antifolate described herein that is sufficient to achieve the effect of the intended application including, but not limited to, disease treatment, as defined below. The therapeutically effective amount may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells. The specific dose will vary depending on the particular antifolate (i.e. compound) chosen, the dosing regimen to be followed, whether it is administered in combination with other compounds, route and timing of administration, the tissue to which it is administered, and the physical delivery system in which it is carried.

The terms “treatment” and “treating” refer to an approach for obtaining beneficial or desired results including, but not limited to, therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

The term “therapeutic effect,” as that term is used herein encompasses a direct and/or an indirect therapeutic benefit and/or a prophylactic benefit as described above. A direct therapeutic benefit may include relief from a disease condition, such as a viral infection, by direct inhibition or mitigation of the cause(s) of such disease, such as in the case of a viral infection, the inhibition of viral replication. An indirect therapeutic benefit may include alleviation of certain manifestations of a disease that is not universal or granted, but may appear for specific patients, or when certain conditions are met. For example, in case of a severe immunogenic response to a viral infection in certain patients in progressed forms of the disease leading to sepsis and multiorgan failure, the benefit of the current invention would be from its indirect suppression of the intensity of such immunogenic response to prevent sepsis. A prophylactic effect includes delaying or eliminating the appearance of a disease or condition, delaying or eliminating the onset of symptoms of a disease or condition, slowing, halting, or reversing the progression of a disease or condition, or any combination thereof.

The term “subject” or “patient” refers to an animal, such as a mammal, for example a human. The methods described herein can be useful in both human therapeutics and veterinary applications. In some embodiments, the patient is a mammal, and in preferred embodiments, the patient is human. For veterinary purposes, the term “subject” and “patient” include, but are not limited to, farm animals including cows, sheep, pigs, horses, and goats; companion animals such as dogs and cats; exotic and/or zoo animals; laboratory animals including mice, rats, rabbits, guinea pigs, and hamsters; and poultry such as chickens, turkeys, ducks, and geese.

The term “alkyl” refers to any substituted or unsubstituted alkane missing one hydrogen.

The term “alkenyl” refers to any substituted or unsubstituted alkene missing one hydrogen. The term “alkynyl” refers to any substituted or unsubstituted alkyne missing one hydrogen.

The term “d90 particle size” refers to the particle size in which 90% of particles have a diameter less than that size. The d90 particle size may be determined using light scattering or automatic microscopical imaging analysis.

The term “targeted drug delivery” refers to any method of drug delivery that increases the concentration of the medication in some parts of the body relative to the others.

The term “ready-to-administer aqueous solution” refers to an aqueous solution that can be directly administered to a patient (e.g. with little or no compounding and preparation), and/or wherein no dilution is required before being administered to the patient. The ready to administer aqueous solution may be used for direct injection, for example, subcutaneous injection, intravenous injection, intramuscular injection, or intradermal injection.

The term “ready-to-dilute” aqueous solution refers to an aqueous solution that can be administered after dilution, for example, with a pharmaceutically acceptable solvent or intravenous fluid medium.

The term “good manufacturing practice” or “GMP” refer to the guidelines established by the World Health Organization and adopted by various regulatory bodies for ensuring that pharmaceutical products are consistently produced and controlled according to quality standards that are designed to minimize the risks involved in any pharmaceutical production that cannot simply be eliminated through the testing of the final product. GMP covers all aspects of production, raw materials, premises and equipment to the training and personal hygiene of staff. Various health-regulatory agencies, e.g. FDA, EMIR that control the authorization and licensing of the manufacture and sale of pharmaceuticals require that pharmaceutical production conforms to such GMP guidelines.

The term “Osmolality” is used to describe the tonicity or the osmotic pressure of a solution. Osmolality is influenced by the concentration of solutes in the solution and is often measured by the extent of lowering (or depression) of freezing point of the solution due to such dissolved matter. Osmolality is considered a key attribute of injections to ensure that the blood plasma, the fluid inside blood cells and the membranes of the blood vessels where the drug is administered have the same or similar osmotic pressure as the injected solution. A substantial change in osmotic pressure following the administration of such injection could otherwise cause damage due to build of pressure across local cellular membranes and/or rapid flux of fluids through the membranes to re-establish osmotic equilibrium. Osmotic pressure in this document is represented by the unit milliosmole (mOsm), which is equivalent to and interchangeable with mOsm/L.

Any method of treating a disease described herein, the method comprising administering to a subject in need thereof a therapeutically effective amount of a medicament may be rephrased or reformulated as “a medicament for use in the treatment of a disease”. In other words, if the disease is a viral pulmonary infection and the medicament is an antifolate the method of treatment can be rephrased to “an antifolate for use in the treatment of a viral pulmonary infection”. If the disease is lung cancer and the medicament is a pharmaceutical formulation, the method can be reformulated as “a pharmaceutical formulation for use in the treatment of lung cancer”.

Antifolate

According to any aspect of the present invention, an antifolate refers to any compound, or a metabolite thereof, that inhibits the folate dependent pathways (e.g. which may be a compound that blocks the action of folic acid (vitamin B9) in the cell). In some embodiments, the antifolate, or metabolite thereof, may be an inhibitor of one or more enzymes in the folate-dependent pathway.

In some embodiments, the antifolate, or a metabolite thereof, is a dihydrofolate reductase (DHFR) inhibitor. In some embodiments, the antifolate, or a metabolite thereof, is a thymidylate synthase (TS) inhibitor. In some embodiments, the antifolate, or a metabolite thereof, is a glycinamide ribonucleotide transferase (GARFT) inhibitor. In some embodiments, the antifolate, or a metabolite thereof, is a dihydrofolate reductase (DHFR) inhibitor, a thymidylate synthase (TS) inhibitor and/or a glycinamide ribonucleotide transferase (GARFT) inhibitor. In certain embodiments, the antifolate metabolite may be a glutamated antifolate. In some examples, the antifolate metabolite may be a mono, di or poly glutamated antifolate. A poly-glutamated antifolate may refer to an antifolate comprising at least three glutamates (i.e. which has been glutamated at least three times), or at least four glutamates, or at least five glutamates, or six or more glutamates. In some embodiments, the antifolate may be metabolized in the cell by folylpolyglutamate synthetase (FPGS) to form the glutamated antifolate (e.g. a mono, di, or poly glutamated antifolate). In certain embodiments, the glutamated antifolate (e.g. mono, di, or poly glutamated antifolate) may be a better inhibitor of at least one of dihydrofolate reductase (DHFR), thymidylate synthase (TS) and/or glycinamide ribonucleotide transferase (GARFT) as compared to the antifolate. For example, for the antifolate pemetrexed, polyglutamated pemetrexed (even more specially pentaglutamated pemetrexed) is a better inhibitor of DHFR than pemetrexed.

In certain embodiments, the antifolate may inhibit the synthesis of DNA or RNA nucleotides. In certain embodiments, the antifolate may inhibit the synthesis of one or more ribonucleotides selected from guanosine and adenosine. In certain embodiments, the antifolate may inhibit the synthesis of one or more deoxyribonucleotides selected from thymidine, deoxyadenosine, and deoxyguanosine.

In certain embodiments, the antifolate is a compound of formula (I), or a pharmaceutically acceptable salt thereof:

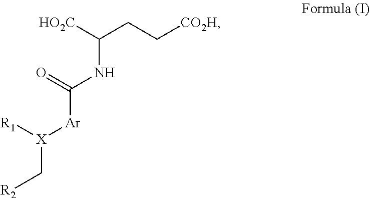

wherein Ar is selected from a 5-membered aromatic ring or 6-membered aromatic ring;
wherein X is selected from N or CH
wherein R₁ is selected from H, alkyl, alkenyl or alkynyl and
wherein R₂ is selected from

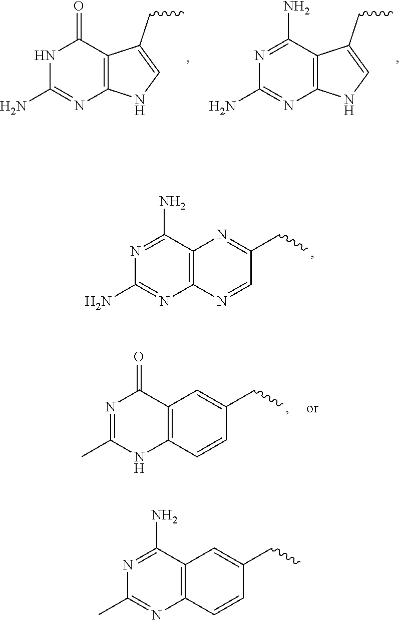

In a certain embodiment, the compound of Formula (I), or a pharmaceutically acceptable salt thereof, has the following stereochemistry:

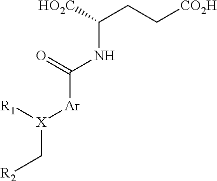

In certain embodiments, R₁ may be selected from H, methyl or propargyl.

In some embodiments, the 5-membered aromatic ring may be selected from furan, thiophene, pyrrole, imidazole, oxazole, thiazole or furazan. In a preferred embodiment, the 5-membered aromatic ring is thiophene.

In an embodiment, the 6-membered aromatic ring is selected from benzene, pyridine, pyrazine, pyrimidine or pyridazine. In a preferred embodiment, the 6-membered aromatic ring is benzene.

In an embodiment, Ar may be selected from

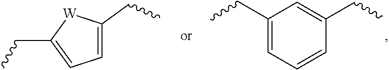

wherein W is selected from N, O or S, preferably S.

In a certain embodiment, the compound of formula (I) may be selected from one of the following structures:

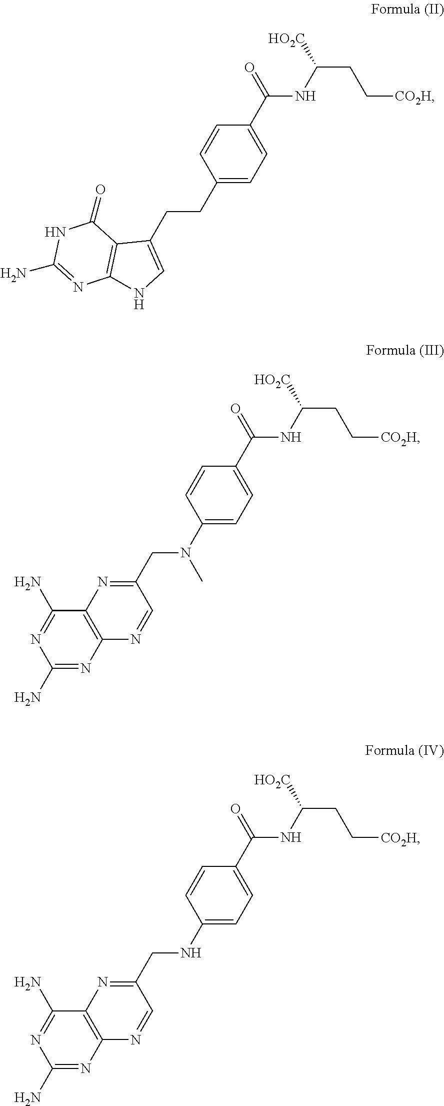 [SMILES: Nc1nc2[nH]cc(CCc3ccc(C(=O)N[C@@H](CCC(=O)O)C(=O)O)cc3)c2c(=O)[nH]1 ; CN(Cc1cnc2nc(N)nc(N)c2n1)c1ccc(C(=O)N[C@@H](CCC(=O)O)C(=O)O)cc1 ; Nc1nc(N)c2nc(CNc3ccc(C(=O)N[C@@H](CCC(=O)O)C(=O)O)cc3)cnc2n1]

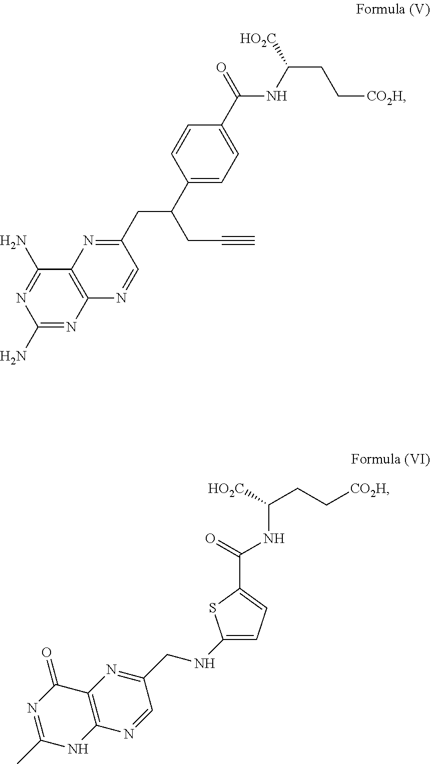 [SMILES: C#CCC(Cc1cnc2nc(N)nc(N)c2n1)c1ccc(C(=O)N[C@@H](CCC(=O)O)C(=O)O)cc1 ; Cc1nc(=O)c2nc(CNc3ccc(C(=O)N[C@@H](CCC(=O)O)C(=O)O)s3)cnc2[nH]1]

or pharmaceutically acceptable salts thereof.

In a certain embodiment, the antifolate is a compound of formula (II), or a pharmaceutically acceptable salt thereof:

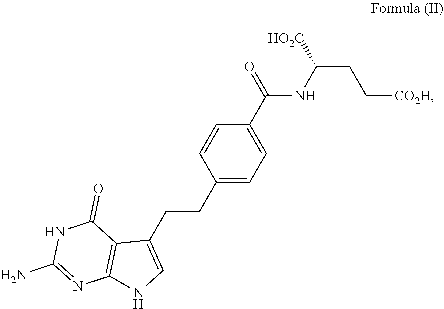 [SMILES: Nc1nc2[nH]cc(CCc3ccc(C(=O)N[C@@H](CCC(=O)O)C(=O)O)cc3)c2c(=O)[nH]1]

The compound of formula (II) is otherwise known as pemetrexed in the art. In some embodiments, the pemetrexed may be metabolized in the cell to form glutamated pemetrexed, for example, pentaglutamated pemetrexed.

In certain embodiments, the compounds of formula (I), (II), (Ill), (IV), (V) or (VI) may be selected from the free acid or a salt, wherein the salt is selected from aluminum, arginine benzathine, chloroprocaine, calcium, choline, diethanolamine, ethanolamine, ethylenediamine, lysine, magnesium, lithium, histidine, sodium, potassium, tromethamine, meglumine, procaine, triethylamine or zinc. In a preferred embodiment, the compounds of (I), (II), (Ill), (V), or (VI) may be selected from sodium, or potassium or tromethamine salt forms.

Certain salts, including the disodium salt form, are found to improve cellular uptake in the lungs, for example, in lung alveolar cells, for example, type II lung alveolar cells.

Method of Treating a Viral Pulmonary Infection

According to the first aspect, there is provided a method of treating a viral pulmonary infection, the method comprising administering to a subject in need thereof a therapeutically effective amount of an antifolate. The antifolate may be any antifolate as described herein. A viral pulmonary infection may be any infection caused by a virus in the lungs or in the inner airways leading into the lung.

In certain embodiments, the antifolate is found to slow or halt viral replication. This may be achieved by blocking or slowing the synthesis of DNA or RNA nucleotides in the infected cell, such that the virus does not have the building blocks to replicate their genome.

In certain embodiments, the antifolate is also found to have an immunosuppressive effect. This reduces the inflammatory response and sepsis in response to the viral pulmonary infection.

In some embodiments, the viral pulmonary infection may cause viral pneumonia. In some embodiments, the viral pulmonary infection may cause bronchiolitis.

In some embodiments, the viral pulmonary infection may be caused by an RNA virus or a DNA virus. For example, in one embodiment, the viral pulmonary infection is caused by an RNA virus. In some embodiments, the viral pulmonary infection is caused by an RNA virus with at least 10 kilobases, or at least 13 kilobases, or at least 20 kilobases, or at least 30 kilobases.

In some cases, the method of treatment may be more effective for viral pulmonary infections caused by RNA viruses with larger genomes due to a depletion of RNA nucleotides in the cell.

In certain embodiments, the viral pulmonary infection is a coronavirus infection, an influenza infection or a respiratory syncytial viral (RSV) infection.

The influenza infection may be caused by any type or strain of influenza. In some embodiments, the influenza infection may be selected from Influenza A, Influenza B, Influenza C or Influenza D. In some embodiments, the influenza A infection may be selected from H1N1, H2N2, H3N2, H5N1, H7N7, H1N2, H9N2, H7N2, H7N3, H10N7, H7N9 or H6N1.

The coronavirus infection may be caused by any type or strain of coronavirus. In some embodiments, the coronavirus infection may be an alphacoronavirus infection or a betacoronavirus infection, preferably a betacoronavirus. The betacoronavirus may have an A lineage, a B lineage, a C lineage or a D lineage, for example, a B lineage. In preferred embodiments, the coronavirus infection may be COVID-19, otherwise known as SARS-CoV-2 or 2019-nCoV.

In an embodiment, the antifolate is administered orally, parenterally, by inhalation or by nebulization, or by intratracheal instillation. In a preferred embodiment, the antifolate is administered such that there is targeted or selective, preferential distribution of the antifolate to the lung, for example, to lung alveolar cells, for example, type II lung alveolar cells.

In a preferred embodiment, the antifolate is administered parenterally to lung alveolar cells, for example, type II lung alveolar cells. The antifolate may be administered parenterally by injection, for example, by intravenous injection, or by intradermal injection, preferably as an intravenous bolus injection, by intramuscular injection or by subcutaneous injection. Subcutaneous injection can be particularly advantageous because it is often non-intrusive, safe, well-tolerated, and/or can have reduced resource use due to reduced need for specialized skills or monitoring during administration. In some embodiments, the antifolate may be administered by an intravenous bolus injection in multiple doses. This may have the effect of extending the plasma levels without the need to use high doses of the active component.

In another preferred embodiment, the antifolate is administered by intratracheal instillation to the lung.

In an embodiment, the antifolate is administered every 4 hours up to every 4 weeks, or from 3 to 4 hours up to every 2 weeks. In another embodiment, the antifolate is administered up to every 3 hours, up to 4 hours, or up to every 8 hours, or up to every 12 hours, or up to every 16 hours, or up to every 24 hours, or up to every 48 hours, or up to every 36 hours, or up to every 72 hours, or up to every 144 hours, or up to one week, or up to once every 2 weeks, or up to once every 3 weeks, or up to once every 4 weeks. The antifolate may be administered more regularly if the symptoms are more severe. An effective amount of the antifolate may be administered in either single or multiple doses (e.g., twice, three times, four times, five times or even six times a day). In an example, the antifolate may be administered in multiple doses between 2 hours and 4 hours apart, for example, about 3 hours apart. The multiple doses may comprise a dosage of from about 1 to about 200 mg, or from about 35 mg to about 175 mg, or from about 50 mg to about 150 mg.

In an embodiment, the antifolate is administered immediately after infection (e.g. infection with SARS-CoV-2), or at least 1 day after infection, or at least 2 days after infection, or at least 3 days after infection. In an embodiment, the antifolate is administered immediately after testing positive for a viral pulmonary infection (e.g. SARS-CoV-2 infection). In another embodiment, the antifolate is administered after the onset of one or more symptoms. The one or more symptoms may include coughing, fever, difficulty of breathing, aches, loss of smell and/or taste, fatigue, or a combination thereof. In an embodiment, the antifolate is administered within 6 hours of onset of one or more symptoms, or within 12 hours, or within 24 hours, or within 48 hours, or within 72 hours after the onset of one or more symptoms. In an embodiment, the antifolate is administered immediately after hospitalization from viral pulmonary infection. In another embodiment, the antifolate is administered to hospitalized patients requiring oxygen supplementation in sufficient doses so as to reach sufficient blood plasma concentration of the antifolate that exceeds the EC90 of the antifolate against the infecting virus as determined from an in-vitro cell based assay. The test for the viral pulmonary infection may be one or more of a PCR test, nanopore test or antigen test (e.g. lateral flow test).

The amount of the compound to be administered (i.e. the dosage) is dependent on the mammal being treated, the severity of the disorder or condition, the rate of administration, the disposition of the compound and the discretion of the prescribing physician. In an embodiment, the antifolate dosage is from about 0.01 mg/m² to about 700 mg/m² of body surface area, or from 0.05 mg/m² to 300 mg/m², or from about 0.1 mg/m² to 150 mg/m² or from about 25 to about 100, or from about 35 mg/m² to about 75 mg/m², or from about 45 mg/m² to about 55 mg/m² of body surface area. In an embodiment, the antifolate dosage is selected based on the volume of distribution of the antifolate and its concentration in the blood plasma and the target site of infection. In another embodiment, the dose is fixed at about 100 mg for adults and at 50 mg/m². These dosages may be useful for IV bolus injection. In yet another embodiment, the dose is greater than about 0.01 mg/m² of body surface area, or greater than about 0.05 mg/m², or greater than about 0.1 mg/m², or greater than about 0.25 mg/m², or greater than about 0.5 mg/m², or greater than about 1 mg/m², or greater than about 5 mg/m², or greater than about 10 mg/m², or greater than about 25 mg/m², or greater than about 50 mg/m², or greater than about 75 mg/m², or greater than about 100 mg/m², or greater than about 150 mg/m², or greater than about 200 mg/m², or greater than about 250 mg/m², or greater than about 300 mg/m², or greater than about 350 mg/m², or greater than about 400 mg/m², or greater than about 500 mg/m², or greater than about 550 mg/m², or greater than about 600 mg/m², or greater than about 650 mg/m² of body surface area. In an embodiment, the antifolate dosage is less than about 700 mg/m² of body surface area, or less than about 650 mg/m², or less than about 600 mg/m², or less than about 550 mg/m², or less than about 500 mg/m², or less than about 450 mg/m², or less than about 400 mg/m², or less than about 350 mg/m², or less than about 300 mg/m², or less than about 250 mg/m², or less than about 200 mg/m², or less than about 150 mg/m², or less than about 125 mg/m², or less than about 100 mg/m², or less than about 75 mg/m², or less than about 50 mg/m², or less than about 25 mg/m², or less than about 10 mg/m², or less than about 5 mg/m², or less than about 2.5 mg/m², or less than about 1 mg/m², or less than about 0.5 mg/m², or less than about 0.25 mg/m², or less than about 0.1 mg/m² or less than about 0.05 mg/m² of body surface area. The antifolate dosage required for viral respiratory disease may be less than the unit dosage administered for lung cancer, for example, the dosage for viral pulmonary infection may be less than about 500 mg/m², or less than about 100 mg/m², or less than 75 mg/m²′ of body surface area. This may provide an antiviral therapy that exceeds EC90 while keeping the overall exposure and systemic toxicity low.

In some embodiments, the amount of antifolate administered to the subject is from about 0.2 mg to 300 mg, or from about 0.5 mg to 250 mg, or from about 1 mg to 200 mg, or from about 3 mg to about 300 mg, or from about 25 mg to about 175 mg of antifolate, or from about 35 mg to about 175 mg of antifolate, or from about 50 mg to about 150 mg. In some embodiments, the amount of antifolate administered to the subject may be greater than about 0.2 mg of antifolate, or greater than about 0.5 mg of antifolate, or greater than about 1 mg of antifolate, or greater or greater than about 2.5 mg of antifolate, or greater than about 5 mg of antifolate, or greater than 10 mg of antifolate, or greater than 25 mg of antifolate. In some embodiments, the amount of antifolate administered to the subject may be less than about 300 mg of antifolate, or less than about 200 mg of antifolate, or less than about 100 mg of antifolate, or less than about 75 mg of antifolate, or less than about 50 mg of antifolate, or less than 45 mg of antifolate, or less than about 25 mg of antifolate, or less than about 10 mg of antifolate, or less than 5 mg of antifolate, or less than about 1 mg of antifolate, or less than 0.5 mg of antifolate. In some embodiments, the amount of antifolate may be adjusted from the initial loading dose and in subsequent doses to maintain the blood plasma concentration of the antifolate over and above the EC90—if viral infection, for a sufficient duration of time for the antifolate to be taken up by the target tissue at its site of action in sufficient quantities. In an embodiment, the amount of antifolate-administered to the subject is from about 0.2 mg to about 300 mg, or from about 0.5 mg to about 250 mg of antifolate, or from about 1 mg to about 200 mg of antifolate, or from 10 mg to 100 mg of antifolate, or from 15 mg to 75 mg of antifolate. The amount of antifolate administered (i.e. the antifolate dosage) in a unit dose as required for viral respiratory disease may be less than the unit dosage administered for lung cancer, for example, the amount of antifolate administered to a subject for viral pulmonary infection may be less than about 200 mg, or less than about 100 mg, or less than about 80 mg. In an embodiment, the initial loading dose is between about 100 to about 175 mg of antifolate and the subsequent dose is between about 50 and about 100 mg of antifolate.

In an embodiment, the antifolate is administered using any pharmaceutical formulation described herein. In an embodiment, the antifolate is administered using a pharmaceutical formulation comprising the antifolate, sodium sulfite and cysteine hydrochloride. In an embodiment, the pharmaceutical formulation comprising the antifolate sodium sulfite and cysteine hydrochloride is dissolved in water for injection. In another embodiment, the antifolate formulation is an aqueous solution or predominantly an aqueous solution. The pharmaceutical formulation may be administered directly to the lungs. The pharmaceutical formulation may be in the form of an aqueous solution which has excellent stability, as may be described herein. The pharmaceutical formulation may be suitable for sterilization by moist-heat sterilization as by autoclaving during or after manufacturing the product. In some embodiments, the pharmaceutical formulation is a heat-sterilized pharmaceutical formulation. The formulation may be suitable for intratracheal instillation to the lung or for parenteral administration to the lungs, as may be described herein.

In an embodiment of the method of treatment described herein, the antifolate is not administered concurrently or its administration preceded by treatment with folic acid or vitamin B₁₂ (e.g. for multiple days). In an embodiment, the method of treatment does not require co-administration of corticosteroids, such as dexamethasone. In an embodiment, treatment with folic acid or vitamin B₁₂ or their co-therapy may only be initiated after use of more than ordinarily recommended doses of the antifolate to treat aggressive forms of the disease. In an embodiment, the antifolate is not administered concurrently with folic acid or does not require pre-treatment with either folic acid or vitamin B₁₂. This is distinct from methods of treatment using antifolates for lung cancer, wherein antifolates are typically administered with folic acid or B₁₂ to reduce the immunosuppressant effect of antifolates. For treating viral infections, the immunosuppressant effect of antifolates may be effective in reducing sepsis or the inflammatory response.

In some embodiments, the method of treatment may comprise administration by direct injection and the viral pulmonary infection is a coronavirus infection, for example, SARS-CoV-2. The injection may be selected from subcutaneous injection, intravenous injection as a bolus dose or by infusion of a more diluted solution for injection, intramuscular injection, or intradermal injection.

In some embodiments, the method of treatment may comprise administration by direct injection and the antifolate is pemetrexed. The injection may be selected from subcutaneous injection, intravenous injection, intramuscular injection, or intradermal injection.

In some embodiments, the method of treatment may comprise administration by direct injection and the antifolate is provided as any pharmaceutical formulation as described herein. The injection may be selected from subcutaneous injection, intravenous injection (i.e. to be administered either as a bolus injection or as an infusion following dilution, if necessary) intramuscular injection, or intradermal injection.

In some embodiments, the method of treatment may comprise administration by direct injection and the amount of antifolate dosage is from about 25 mg/m² to about 100 mg/m² and optionally from about 35 mg/m² to about 75 mg/m². The injection may be selected from subcutaneous injection, intravenous injection, intramuscular injection, or intradermal injection methods.

In some embodiments, the method of treatment may comprise administration by direct injection and the amount of antifolate administered to the subject is from about 3 to about 300 mg, optionally from about 25 mg to about 175 mg, or optionally from about 50 mg to about 150 mg. The injection may be selected from subcutaneous injection, intravenous injection, intramuscular injection, or intradermal injection methods.

In some embodiments, the antifolate is pemetrexed and/or is provided as a pharmaceutical formulation as described herein, and the amount of antifolate dosage is from 25 mg/m² to 100 mg/m² of body surface area and optionally from about 35 mg/m² to about 75 mg/m² of body surface area.

In some embodiments, the antifolate is pemetrexed and/or is provided as a pharmaceutical formulation as described herein, and the and the amount of antifolate administered to the subject is from about 1 to about 200 mg, optionally from about 25 mg to about 175 mg, or optionally from about 50 mg to about 150 mg.

Pharmaceutical Formulation

According to a second aspect, there is provided a pharmaceutical formulation comprising an antifolate, sodium sulfite, and cysteine hydrochloride. In some embodiments, the pharmaceutical formulation comprises an antifolate, sodium sulfite, cysteine hydrochloride and water for injection. The water for injection may include a suitable source of counter ion to the free acid for in-situ salt formation and to increase solubility, and optionally a pH adjuster to adjust the pH of the final solution. The source of counter ion for pemetrexed free acid could, for example, be sodium hydroxide. It is also understood that the sodium sulfite in the pharmaceutical formulation can be replaced by other commonly used forms, such as, sodium metabisulfite or potassium metabisulfite or sodium bisulfite. The pharmaceutical formulation may comprise an antifolate as described herein. The pharmaceutical formulation according to the second aspect may be used in the method of the first aspect to administer to a subject in need thereof a therapeutically effective amount of an antifolate.

In certain embodiments, the weight ratio of the antifolate to cysteine hydrochloride is from about 12:1 to about 55:1, or from about 13:1 to about 25:1, or about 14:1 to about 18:1, or about 14:1 to 16:1, or about 14:1 to about 15:1, or about 14.5:1. In some embodiments, the weight ratio of the antifolate to cysteine hydrochloride is greater than about 12:1, or greater than about 12.5:1, or greater than about 13:1, or greater than about 14:1. In some embodiments the weight ratio of the antifolate to cysteine hydrochloride is less than about 55:1, or less than about 40:1, or less than about 30:1, or less than about 20:1, or less than about 17.5:1, or less than about 15:1. In a preferred embodiment, the antifolate is a compound of Formula (II), i.e., pemetrexed.

In certain embodiments, the weight ratio of the antifolate to sodium sulfite is from about 2.5:1 to about 22:1, or from about 3:1 to about 15:1, or from about 4:1 to about 9:1, or from about 4:1 to about 4:1 to about 7:1, or about 4:1 to about 6:1, or about 4:1 to about 5:1, or about 4.5:1 to about 5:1, or about 4.8:1 to about 5:1, or about 4.94:1. In a preferred embodiment, the antifolate is a compound of Formula (II), i.e., pemetrexed. In some embodiments, the weight ratio of the antifolate to sodium sulfite is greater than about 2.5:1, or greater than about 3:1, or greater than about 4:1, or greater than about 4.5:1. In some embodiments, the weight ratio of the antifolate to sodium sulfite is less than about 22:1, or less than about 15:1, or less than about 9:1, or less than about 8:1, or less than about 7:1, or less than about 6:1, or less than about 5:1.

In certain embodiments, the weight ratio of cysteine hydrochloride to sodium sulfite is from about 1:1.5 to about 1:4.5, or from about 1:2.5 to 1:4, or from about 1:2.8 to 1:3.1. In certain embodiments, the weight ratio of cysteine hydrochloride to sodium sulfite is greater than about 1:4.5, or greater than about 1:3:5, or greater than about 1:3. In certain embodiments, the weight ratio of cysteine hydrochloride to sodium sulfite is less than about 1:1.5, or less than about 1:2, or less than about 1:2.5.

The weight ratios of antifolate, cysteine hydrochloride and sodium sulfite are such that the pharmaceutical formulation has excellent stability, especially in aqueous solution. The weight ratios of antifolate, cysteine hydrochloride and sodium sulfite are therefore such that the pharmaceutical formulation may have extended shelf-life at room temperature storage, or during brief exposures to elevated temperatures, such as, during sterilization or during shipping and handling

The pharmaceutical formulation has been developed such that the components are present in suitable quantities to best counter the oxidative and hydrolytic degradation of antifolates, such as pemetrexed, i.e., the compound of Formula (II), e.g., when in aqueous solution. As compared to prior art formulations, the pharmaceutical formulation described herein demonstrates increased oxidative stability.

The weight ratios of antifolate, cysteine hydrochloride and sodium sulfite are such that the pharmaceutical formulation has a pH within a desirable range when in aqueous solution.

The pharmaceutical formulation may be in the form of an aqueous solution, a suspension, an emulsion or a powder. In a preferred embodiment, the pharmaceutical formulation is in the form of an aqueous solution. The aqueous solution may be an isotonic and/or sterile aqueous solution. In certain embodiments, the sterile aqueous solution may have been sterilized by any suitable method. In certain embodiments, the sterile aqueous solution may have been sterilized by moist-heat terminal sterilization.

The aqueous solution may have any suitable pH. In preferred embodiments, the aqueous solution has a pH from about 6 to about 7.5, or from about 6.5 to about 7.5, or from about 7 to about 7.5. In preferred embodiments, the aqueous solution has a pH of less than about 7.5, or less than about 7.45, or less than about 7.4, or less than about 7.35, or less than about 7.2, or less than or equal to about 7.25. A pH within this range is believed to reduce the extent of oxidation of the antifolate without causing precipitation, for example, of cystine, an oxidative product of cysteine.

In certain embodiments, the aqueous solution has a dissolved oxygen content of less than 2 ppm, or less than 1 ppm, or less than 0.5 ppm. In certain embodiments, the aqueous solution may further comprise an inert gas. The inert gas may be argon or nitrogen, preferably nitrogen.

In certain embodiments, the aqueous formulation may have a density from about 0.9 g/mL to about 1.2 g/mL, or from about 1.000 g/mL to about 1.02 g/mL

In certain embodiments, the aqueous formulation may have an osmolality from about 150 mOsm to about 400 mOsm, or from 250 mOsm to about 400 mOsm, or from about 150 mOsm to about 300 mOsm, or from about 200 mOsm to about 280 mOsm, or from about 270 mOsm to about 350 mOsm. In some embodiments, the osmolality is greater than 150 mOsm, or greater than 200 mOsm, or greater than 225 mOsm, or greater than 250 mOsm, or greater than 270 mOsm, or greater than 285 mOsm, or greater than 300 mOsm. In some embodiments, the osmolality is less than 400 mOsm, or less than 375 mOsm, or less than 350 mOsm, or less than 325 mOsm. An osmolality between 250 mOsm and 400 mOsm, for example between 270 mOsm and 350 mOsm, can be particularly useful for direct injection.

In certain embodiments, the aqueous formulation may have a viscosity from about 0.35 cP to about 2.5 cP, or about 1 cP.

In some embodiments, the antifolate is present in the aqueous solution at a concentration from about 0.5 mg/mL to about 100 mg/mL in the aqueous solution, or from 0.5 mg/mL to about 50 mg/mL in the aqueous solution or from about 10 mg/mL to about 80 mg/mL, or from 10 mg/mL to about 40 mg/mL in the aqueous solution, or from about 20 mg/mL to about 30 mg/mL in the aqueous solution, or from about 22.5 mg/ml to about 27.5 mg/ml in the aqueous solution, for example about 25 mg/mL in the aqueous solution. In yet another other embodiment, the antifolate is present in the aqueous solution at a concentration from about 35 mg/mL to about 80 mg/mL in the aqueous solution, or from about 40 mg/mL to about 55 mg/mL in the aqueous solution, or from about 60 mg/mL to about 80 mg/mL. In some embodiments, the antifolate is present in the aqueous solution at a concentration greater than 0.5 mg/mL in the aqueous solution, or greater than 1 mg/mL, or greater than 5 mg/mL, or greater than 10 mg/mL, or greater than 20 mg/mL, or greater than 30 mg/mL in the aqueous solution, or greater than 40 mg/mL, or greater than 50 mg/mL in the aqueous solution. At such a concentration, the antifolate may be suitable for administration by injection, e.g., subcutaneous or intravenous bolus injection. In some embodiments, the antifolate is present in the aqueous solution at a concentration from about 0.5 mg/mL to about 20 mg/mL or from about 25 mg/mL to about 50 mg/mL, or from about 0.5 mg/mL to about 100 mg/mL. At such a concentration, the antifolate may be suitable for administration by intravenous infusion and/or by direct bolus intravenous injection. The concentration of the antifolate in the pharmaceutical formulation may depend on how the antifolate is administered and/or the disease that is being treated. For example, the concentration of the antifolate in the pharmaceutical formulation may be higher for viral therapy as compared to cancer therapy. In an example, the concentration of antifolate in the pharmaceutical formulation is about 75 mg/mL. A high concentration of antifolate can allow for low dose-volume.

In some embodiments, the antifolate is present in the aqueous solution in an amount of from about 2% w/w to about 10% w/w based on the total amount of aqueous solution, or about 2% w/w to about 8% w/w based on the total amount of aqueous solution, or from about 2.19% w/w to about 2.5% w/w or from about 4.0 to 5.5% w/w based on the total weight of the aqueous solution. In some embodiments, the cysteine hydrochloride monohydrate is present in the aqueous solution in an amount of from about 0.04% w/w to about 0.2% w/w based on the total amount of aqueous solution. In some embodiments, the sodium sulfite is present in an amount of about 0.1% w/w to about 0.95% w/w based on the total amount of aqueous solution. In some embodiments, the aqueous solution comprises NaOH in an amount from about 0.4% w/w to about 0.9% w/w based on the total amount of the aqueous solution. In some embodiments, the aqueous solution comprises about 2% w/w to about 10% w/w of antifolate, 0.04% w/w to about 0.2% w/w cysteine hydrochloride monohydrate and 0.1% w/w to about 0.95% w/w sodium sulfite based on the total amount of aqueous solution and optionally 0.4% w/w to about 0.9% w/w NaOH based on the total amount of the aqueous solution. In some embodiments, the aqueous solution comprises 94% w/w to 97.9% w/w water based on the total amount of the aqueous solution. A source of NaOH may be used when the free acid pemetrexed is used, as opposed to the disodium salt form.

In some embodiments, the aqueous solution is a ready-to-administer sterile aqueous solution. In some embodiments, the aqueous solution of the pharmaceutical formulation is a diluted aqueous solution diluted from a ready-to-dilute aqueous solution.

In some embodiments, the ready-to-administer aqueous solution or the diluted aqueous solution is suitable for parenteral administration, for example, parenteral administration to the lungs. The ready to administer solution or the diluted aqueous solution may be suitable for direct injection, for example, as an intravenous injection or subcutaneous injection.

In some embodiments, the ready-to-administer aqueous solution comprises, or the ready-to-dilute aqueous solution is diluted with, a pharmaceutically acceptable solvent or intravenous fluid medium. In some embodiments, the pharmaceutically acceptable solvent or intravenous fluid medium may be selected from water (for injection), sodium chloride (Saline) and dextrose, for example, 5% dextrose (D5W). In some embodiments, the pharmaceutically acceptable solvent or intravenous fluid medium is an aqueous solution comprising 0.8 wt. % to about 1.0 wt. % sodium chloride, or about 0.9 wt. % sodium chloride. In some embodiments, the pharmaceutically acceptable solvent or intravenous fluid medium may be free of calcium. The presence of calcium is found, in some cases, to increase the extent of antifolate precipitation.

In certain embodiments, the antifolate is present in the ready-to-dilute aqueous solution at a concentration of from about 1 mg/mL to about 100 mg/mL, or from about 5 mg/mL to about 75 mg/mL, or from about 10 mg/mL to about 40 mg/mL, or from about 20 mg/mL to about 40 mg/mL In some embodiments, the antifolate is present in the ready-to-dilute aqueous solution at a concentration greater than about 1 mg/mL, or greater than about 5 mg/mL, or greater than about 10 mg/mL, or greater than about 20 mg/mL, or greater than or equal to about 25 mg/mL. In some embodiments, the antifolate is present in the ready-to-dilute aqueous solution at a concentration less than about 100 mg/mL, or less than about 75 mg/mL, or less than about 50 mg/mL, or less than about 40 mg/mL, or less than about 35 mg/mL, or less than about 30 mg/mL, or less than or equal to about 25 mg/mL.

In some embodiments, the ready-to-administer aqueous solution or the diluted aqueous solution may be formed by diluting the ready-to-dilute solution at least 2 times to about 10 times, or at least 3 times to about 7 times, or at least 4 times to about 6 times, or about 5 times. In some embodiments, the ready-to-administer aqueous solution or the diluted aqueous solution may be formed by diluting the ready-to-dilute solution by at least 2 times, or at least 2.5 times, or at least 3 times, or at least 3.5 times, or at least 4 times, or at least 4.5 times, or at least 5 times, or at least 6 times, or at least 7 times, or at least 8 times, or at least 9 times, or at least 10 times. In some embodiments, the ready-to-administer aqueous solution or the diluted aqueous solution may be formed by diluting the ready-to-dilute solution by less than about 10 times, or less than about 9 times, or less than about 8 times, or less than about 7 times, or less than about 6 times, or less than about 5 times, or less than about 4 times, or less than about 3 times.

In some embodiments, the aqueous solution is an aerosolized aqueous solution. The aerosolized aqueous solution may be formed using any suitable method, for example, a hand-held mechanical or gas-pressurized nebulizer. The aerosolized aqueous solution may be suitable for inhalation or intratracheal instillation. In some embodiments, the aerosolized aqueous solution has a d90 particle size of less than about 10 μm, or less than about 7.5 μm, or less than about 5 μm. In some embodiments, the aerosolized solution has a d90 particle size from about 0.1 μm to about 10 μm, or from about 0.1 μm to about 7.5 μm, or from about 0.1 μm to about 5 μm. The aerosolized aqueous solution with the particle size according to the present disclosure may aid delivery of the antifolate to the lungs.

In some embodiments, the antifolate may be present in the aerosolized aqueous solution at a concentration from about 0.5 mg/mL to about 50 mg/mL, or from about 0.5 mg/mL to about 20 mg/mL, or from about 0.5 mg/mL to about 10 mg/mL, or from about 0.5 mg/mL to about 5 mg/mL.

In certain embodiments, the pharmaceutical formulation may further comprise one or more pharmaceutically acceptable excipients selected from chelating agents, amino acids, bulking agents, mucolytic agents, buffers, pH and tonicity adjusters, and combinations thereof.

In some embodiments, the chelating agent may be selected from EDTA and salts thereof, citric acid, malic acid, malonic acid, oxalic acid, succinic acid, tartaric acid or a combination thereof.

In some embodiments, the amino acid may be selected from histidine, glutamic acid, lysine, arginine, or a combination thereof.

In some embodiments, the bulking agent may be selected from dextrose, sucrose, mannose, mannitol or a combination thereof.

In some embodiments, the mucolytic agent may be N-acetyl-cysteine. Mucolytic agents may aid delivery of the pharmaceutical formulation to the site of infection in the lungs, by improving permeability and access to the infection across mucous barriers.

In some embodiments, the buffer may be selected from hydrochloric acid, sodium hydroxide, tris, acetate, citrate, tartrate, phosphate, benzoate, bicarbonate, sodium chloride, potassium chloride or a combination thereof.

The pH adjuster may include any suitable acid or base. In an embodiment, the pH adjuster is HCl acid (hydrochloric acid) or NaOH (sodium hydroxide) solution.

In some embodiments, the tonicity adjusters comprise one or more sugars. In some embodiments, the one or more sugars are selected from glucose or dextrose. In some embodiments, the tonicity adjusters comprise one or more salts. In some embodiments, the one or more salts are selected from NaCl or KCl. In some embodiments, the tonicity adjuster is selected from NaCl, KCl, mannitol, glycerin, glucose or dextrose.

In some embodiments, the pharmaceutical formulation may be free of co-solvents, such as propylene glycol. This is advantageous since certain co-solvents, such as propylene glycol, are known to cause nervous system toxicity, localized hemolysis, cardiac arrhythmia, seizures, agitation, and lactic acidosis (e.g. in younger patients or in those with renal or hepatic insufficiency). In some embodiments, the pharmaceutical formulation may be free of chelating agents, such as citric acid.

The pharmaceutical formulations, ready-to-administer aqueous solutions and the ready-to-dilute aqueous solutions described herein may have a shelf-life of at least 6 months, or at least 1 year, or at least 2 years, or at least 3 years when stored under USP controlled room temperature conditions. The antifolate may show less than less than 12.5% degradation, or less than 10% degradation, or less than 8.5% degradation, or less than 6.25% degradation, or less than 2.5% degradation (i.e. as compared to the area under the peak absorbance curve of the active in the chromatogram generated from a standard solution of the active ingredient as measured using HPLC) after at least 6 months, or at least 1 year, or at least 2 years.

The pharmaceutical formulations described may be administered directly (e.g. from the ready-to administer aqueous solution), or can be formed easily, for example, by diluting a ready-to-dilute aqueous solution. This is advantageous since this avoids steps involved in the reconstitution of the lyophilized (freeze-dried) powder, which can compromise sterility, be both more costly and time-consuming. The risks of inadvertent exposure and cytotoxicity to the preparer are also significantly reduced by eliminating reconstitution and/or dilution.

In certain embodiments, the pharmaceutical formulation, ready-to-dilute aqueous solution and/or ready-to-administer aqueous solutions described herein may be provided in any suitable container. In some embodiments, the pharmaceutical formulation, ready-to-administer aqueous solution or the ready-to-dilute aqueous solution described herein may be provided in a glass vial. In some embodiments, the glass vial may be purged with an inert gas, for example, nitrogen. This may increase the shelf-life of the pharmaceutical formulation, ready-to-administer aqueous solution or the ready-to-dilute aqueous solution.

In some embodiments, the pharmaceutical formulation, and/or ready-to-administer aqueous solution and/or ready-to-dilute aqueous solution described herein may be packaged in a single dose unit container or in a multidose primary package and may comprise from about 50 mg to about 1000 mg of antifolate, or from about 75 mg to about 750 mg of antifolate, or from about 100 mg to about 500 mg of antifolate. In some embodiments, the pharmaceutical formulation and/or ready-to-dilute aqueous solution described herein may comprise greater than about 50 mg of antifolate, or greater than about 100 mg of antifolate, or greater than about 250 mg of antifolate, or greater or greater than about 350 mg of antifolate, or greater than about 500 mg of antifolate. In some embodiments, the amount of pharmaceutical formulation and/or ready-to-dilute aqueous solution described herein contained in a single unit or a multidose container may comprise less than about 1000 mg of antifolate, or less than about 750 mg of antifolate, or less than about 650 mg of antifolate, or less than about 500 mg of antifolate, or less than about 400 mg of antifolate, or less than about 300 mg of antifolate, or less than about 200 mg of antifolate. For the multidose container, an additional suitable preservative to prevent microbial contamination from multiple uses, may be added to the formulation composition described herein.

In some embodiments, the pharmaceutical formulation, and/or ready-to-administer aqueous solution and/or ready-to-dilute aqueous solution described herein may be packaged in a primary container composed of type-I glass, such as a type 1 clear glass vial with a rubber stopper and a crimp-cap, and comprise from about 0.2 mg to about 300 mg of antifolate, or from about 0.5 mg to about 250 mg of antifolate, or from about 1 mg to about 200 mg of antifolate based on the total weight of the formulation or solution. In some embodiments, the pharmaceutical formulation and/or ready-to-dilute aqueous solution packaged in the primary container described herein may comprise greater than about 0.2 mg of antifolate, or greater than about 0.5 mg of antifolate, or greater than about 1 mg of antifolate, or greater or greater than about 2.5 mg of antifolate, or greater than about 5 mg of antifolate, or greater than 10 mg of antifolate, or greater than 25 mg of antifolate based on the total weight of the formulation or solution. In some embodiments, the pharmaceutical formulation and/or ready-to-dilute aqueous solution described herein may comprise less than about 300 mg of antifolate, or less than about 200 mg of antifolate, or less than about 100 mg of antifolate, or less than about 75 mg of antifolate, or less than about 50 mg of antifolate, or less than about 25 mg of antifolate, or less than about 10 mg of antifolate, or less than 5 mg of antifolate based on the total weight of the formulation or solution. The antifolate unit dosage required for viral respiratory disease may be less than the unit dosage administered for providing a therapeutic effect for lung cancer.

In some embodiments, the antifolate is pemetrexed and/or the osmolality of the pharmaceutical formulation is from about 250 mOsm to about 400 mOsm. These embodiments may be used in the first aspect of the invention.

In some embodiments, the antifolate is pemetrexed and the pharmaceutical formulation comprises a tonicity adjuster, wherein the tonicity adjuster may be selected from NaCl, KCl, glucose, dextrose, mannitol or glycerin. These embodiments may also be used in the first aspect of the invention.

In some embodiments, the osmolality of the pharmaceutical formulation is from about 250 mOsm to about 400 mOsm and the weight ratio of cysteine hydrochloride to sodium sulfite is from about is from about 1:1.5 to about 1:4.5, or from about 1:2.8 to about 1:3.1. These embodiments may be used in the first aspect of the invention.

In some embodiments, the antifolate is pemetrexed and the weight ratio of cysteine hydrochloride to sodium sulfite is from about is from about 1:1.5 to about 1:4.5, or from about 1:2.8 to about 1:3.1. These embodiments may be used in the first aspect of the invention.

In some embodiments, the antifolate is pemetrexed and the antifolate is present in the pharmaceutical formulation at a concentration from about 0.5 mg/mL to about 100 mg/mL in the pharmaceutical formulation, or from about 10 mg/mL to about 80 mg/mL in the pharmaceutical formulation, or from about 20 mg/mL to about 75 mg/mL in the pharmaceutical formulation, or from about 22.5 mg/ml to about 27.5 mg/ml or from about 45 mg/mL to about 55 mg/mL in the pharmaceutical formulation, or from about 60 or 80 mg/mL in the pharmaceutical formulation. These embodiments may be used in the first aspect of the invention.

In some embodiments, the osmolality of the pharmaceutical formulation is from about 250 mOsm to about 400 mOsm and the antifolate is present in the pharmaceutical formulation at a concentration from about 0.5 mg/mL to about 100 mg/mL in the pharmaceutical formulation, or from about 10 mg/mL to about 80 mg/mL, or from about 20 mg/mL to about 75 mg/mL, or from about 22.5 mg/ml to about 27.5 mg/ml, or from about 45 mg/mL to about 55 mg/mL, or from about 60 mg/ml to about 80 mg/ml for in the pharmaceutical formulation. These embodiments may be used in the first aspect of the invention.

In some embodiments, the antifolate is present in the aqueous solution in an amount of from about 2% w/w to about 10% w/w based on the total amount of aqueous solution, or about 2% w/w to about 8% w/w based on the total amount of aqueous solution, or from about 2.19% w/w to about 2.5% w/w or from about 4.0 to 5.5% w/w based on the total weight of the aqueous solution. In some embodiments, the cysteine hydrochloride monohydrate is present in the aqueous solution in an amount of from about 0.04% w/w to about 0.2% w/w based on the total amount of aqueous solution, or from 0.1% w/w to 0.2% w/w based on the total amount of aqueous solution. In some embodiments, the sodium sulfite is present in an amount of about 0.1% w/w to about 0.95% w/w based on the total amount of aqueous solution, or from 0.3% w/w to about 0.8% w/w based on the total amount of aqueous solution. In some embodiments, the aqueous solution comprises NaOH which is present in an amount from about 0.4 w/w % to about 0.9% w/w based on the total amount of the aqueous solution. In some embodiments, the aqueous solution comprises about 2% w/w to about 10% w/w of antifolate, 0.04% w/w to about 0.2% w/w cysteine hydrochloride monohydrate and 0.1% w/w to about 0.95% w/w sodium sulfite based on the total amount of aqueous solution and optionally 0.4% w/w to about 0.9% w/w NaOH based on the total amount of the aqueous solution. In some embodiments, the aqueous solution comprises 94% w/w to 97.9% w/w water based on the total amount of the aqueous solution. In preferred embodiments, the antifolate is pemetrexed. In some preferred embodiments, the osmolality of the pharmaceutical formulation is from about 250 mOsm to about 400 mOsm.

Also disclosed herein is a pharmaceutical formulation comprising an antifolate, cysteine hydrochloride and at least one of sodium metabisulfite, potassium metabisulfite, sodium bisulfite or sodium sulfite. Also disclosed herein is a pharmaceutical formulation comprising an antifolate; cysteine hydrochloride; at least one of sodium metabisulfite, potassium metabisulfite, sodium bisulfite, or sodium sulfite; and water. This pharmaceutical formulation may be used in the first aspect.

Also disclosed herein is a method of forming the pharmaceutical formulation, the method comprising dissolving the antifolate, cysteine hydrochloride and sodium sulfite in water to form an aqueous solution. In an embodiment, the aqueous solution is filled into a vial, preferably under inert or nitrogen conditions. In an embodiment, the pharmaceutical formulation is sterilized by moist heat (i.e. steam-sterilized). The steam-sterilization may occur at a temperature between 100 and 150° C., for example, between 115° C. to 130° C., for example, about 121° C. In an embodiment, the sterilization is performed for about 12 to about 30 minutes.

Method of Treating Lung Cancer and Other Indications

In a third aspect, there is provided a method of treating lung cancer, the method comprising administering to a subject in need thereof any pharmaceutical formulation as described herein, i.e., a pharmaceutical formulation comprising an antifolate, cysteine hydrochloride and sodium sulfite. In an embodiment the pharmaceutical formulation is in the form of an aqueous solution, optionally containing a source of counterion for the free acid of the antifolate and a pH adjuster. In an embodiment, the lung cancer is non-small cell lung cancer.

In a fourth aspect, there is provided a method of treating mesothelioma, the method comprising administering to a subject in need thereof any pharmaceutical formulation as described herein, i.e., a pharmaceutical formulation comprising an antifolate, cysteine hydrochloride and sodium sulfite. In an embodiment the pharmaceutical formulation is in the form of an aqueous solution, optionally containing a source of counterion for the free acid of the antifolate and a pH adjuster.

In a fifth aspect, there is provided a method of treating an auto immune disorder, the method comprising administering to a subject in need thereof any pharmaceutical formulation as described herein, i.e., a pharmaceutical formulation comprising an antifolate, cysteine hydrochloride and sodium sulfite. In an embodiment the pharmaceutical formulation is in the form of an aqueous solution, optionally containing a source of counterion for the free acid of the antifolate and a pH adjuster.

For any of the above methods, the pharmaceutical formulation may be suitable for targeted delivery to the lungs, as may be described herein. The pharmaceutical formulation may be in the form of an aqueous solution which has excellent stability, as may be described herein. The pharmaceutical formulation may be suitable for inhalation, intratracheal instillation to the lung or for parenteral administration by injection, as may be described herein.

In certain embodiments, the antifolate is found to slow the multiplication of cancer cells. This may be achieved by blocking or slowing the synthesis of DNA or RNA nucleotides in the cancer cell.

In an embodiment, the antifolate is administered orally, parenterally by injection, by inhalation or by nebulisation, or by intratracheal instillation. In a preferred embodiment, the antifolate is administered such that there is targeted delivery of the antifolate to the lung, for example, to lung alveolar cells. Targeted drug delivery refers to any method of drug delivery that increases the concentration of the medication in some parts of the body relative to the others.

In a preferred embodiment, the antifolate is administered parenterally, for example, to lung alveolar cells, for example, type II lung alveolar cells. The antifolate may be administered parenterally by injection, for example, by intravenous injection, by intramuscular injection, by intradermal injection or by subcutaneous injection. Subcutaneous injection can be advantageous because it is often non-intrusive, safe, well-tolerated, and/or requires reduced resource use due to reduced need for specialized skills or monitoring during administration. In some embodiments, the intravenous injection is an intravenous bolus injection. Intravenous bolus injections can have advantages over IV infusions since they are easier and quicker to administer and lead to a rapid availability of the antifolate and/or pharmaceutical composition or formulation in the bloodstream.

In another preferred embodiment, the antifolate is administered by intratracheal instillation to the lung.

In an embodiment, the antifolate is administered every 3 to 4 hours up to every 4 weeks. In an embodiment, the antifolate is administered up to every 3 hours, or up to 4 hours, or up to every 8 hours, or up to every 12 hours, or up to every 16 hours, or up to every 24 hours, or up to every 48 hours, or up to every 36 hours, or up to every 72 hours, or up to every 144 hours, or up to every week, or up to every 2 weeks, or up to once every 3 weeks, or up to once every 4 weeks. The antifolate may be administered depending more regularly if the symptoms are more severe. In an example, the antifolate may be administered in multiple doses between 2 hours and 4 hours apart, for example, about 3 hours apart.

The amount of antifolate that is administered (i.e. the dosage) is dependent on the mammal being treated, the type of infection or cancer being treated, the severity of the disorder or condition, the rate of administration, the condition of the specific patient, any metabolic or genetic abnormalities that might impact the metabolism and elimination of the drug, the disposition of the compound and the discretion of the prescribing physician. In an embodiment, the antifolate dosage is from about 0.01 mg/m² to about 700 mg/m² of body surface area. In an embodiment, the antifolate dosage is greater than about 0.01 mg/m² of body surface area, or greater than about 0.05 mg/m², or greater than about 0.1 mg/m², or greater than about 0.25 mg/m², or greater than about 0.5 mg/m², or greater than about 1 mg/m², or greater than about 5 mg/m², or greater than about 10 mg/m², or greater than about 25 mg/m², or greater than about 50 mg/m², or greater than about 75 mg/m², or greater than about 100 mg/m², or greater than about 150 mg/m², or greater than about 200 mg/m², or greater than about 250 mg/m², or greater than about 300 mg/m², or greater than about 350 mg/m², or greater than about 400 mg/m², or greater than about 500 mg/m², or greater than about 550 mg/m², or greater than about 600 mg/m², or greater than about 650 mg/m² or body surface area. In an embodiment, the antifolate dosage is less than about 700 mg/m² of body surface area, or less than about 650 mg/m² or less than about 600 mg/m², or less than about 550 mg/m², or less than about 500 mg/m² or less than about 450 mg/m², or less than about 400 mg/m², or less than about 350 mg/m² or less than about 300 mg/m², or less than about 250 mg/m², or less than about 200 mg/m² or less than about 150 mg/m², or less than about 125 mg/m², or less than about 100 mg/m² or less than about 75 mg/m², or less than about 50 mg/m², or less than about 25 mg/m², or less than about 10 mg/m², or less than about 5 mg/m², or less than about 2.5 mg/m², or less than about 1 mg/m², or less than about 0.5 mg/m², or less than about 0.25 mg/m², or less than about 0.1 mg/m² or less than about 0.05 mg/m² of body surface area. An effective amount of the antifolate may be administered in either single or multiple doses (e.g., twice or three times a day). In certain embodiments, lower antifolate dosage may be require due to the targeted delivery of the antifolate to the lungs.

In some embodiments, the amount of antifolate administered to the subject is from about 0.2 mg to 300 mg, or from about 0.5 mg to 250 mg, or from about 1 mg to 200 mg. In some embodiments, the amount of antifolate administered to the subject may be less than about 300 mg of antifolate, or less than about 200 mg of antifolate, or less than about 100 mg of antifolate, or less than about 75 mg of antifolate, or less than about 50 mg of antifolate. In some embodiments, the amount of antifolate may be adjusted from the initial loading dose and in subsequent doses to maintain the blood plasma concentration of the antifolate over and above IC90—for cancer, for a sufficient duration of time for the antifolate to be taken up by the target tissue at its site of action in sufficient quantities. In an embodiment, the amount of antifolate-administered to the subject is from about 0.2 mg to about 300 mg, or from about 0.5 mg to about 250 mg of antifolate, or from about 1 mg to about 200 mg of antifolate, or from 10 mg to 100 mg of antifolate, or from 15 mg to 75 mg of antifolate.

In an embodiment, unlike previous methods of treating lung cancer with a therapeutic amount of antifolate, the antifolate may not be administered concurrently with folic acid or does not require pre-treatment with either folic acid or vitamin B12. In some cases, this is because the antifolate dosage may be lower than previous methods, meaning the immunosuppressive effect is lower. This may be due to targeted delivery of the antifolate to the lungs.

In some embodiments, the antifolate is pemetrexed and/or a suitable salt form of pemetrexed and the antifolate is administered parenterally, for example, to lung alveolar cells. In some embodiments, the antifolate is administered parenterally by intravenous infusion. In some embodiments, the antifolate is administered parenterally by injection, for example, by subcutaneous injection, intravenous injection, intramuscular injection, or intradermal injection bolus injection. In some embodiments, the antifolate may be administered by an intravenous bolus injection in multiple doses. This may have the effect of extending the plasma levels without the need to use high doses of the active component.

In some embodiments, the antifolate is pemetrexed and/or the osmolality of the pharmaceutical formulation is from about 250 mOsm to about 400 mOsm.

In some embodiments, the antifolate is pemetrexed and the pharmaceutical formulation comprises a tonicity adjuster, wherein the tonicity adjuster may be selected from NaCl, KCl, glucose, dextrose, mannitol or glycerin.

In some embodiments, the antifolate is pemetrexed and the weight ratio of the antifolate to cysteine hydrochloride is from about 12:1 to about 55:1, or about 13:1 to about 25:1.

In some embodiments, the osmolality of the pharmaceutical formulation is from about 250 mOsm to about 400 mOsm and the weight ratio of the antifolate to cysteine hydrochloride is from about 12:1 to about 55:1, or about 13:1 to about 25:1.

In some embodiments, the osmolality of the pharmaceutical formulation is from about 250 mOsm to about 400 mOsm and the weight ratio of the antifolate to sodium sulfite is from about 2.5:1 to about 22:1, or from about 1:1.5 to about 1:2.5, or from about 4:1 to about 9:1.

In some embodiments, the antifolate is pemetrexed and the weight ratio of the antifolate to sodium sulfite is from about 2.5:1 to about 22:1, or from about 1:1.5 to about 1:2.5, or from about 4:1 to about 9:1.

In some embodiments, the osmolality of the pharmaceutical formulation is from about 250 mOsm to about 400 mOsm and the weight ratio of cysteine hydrochloride to sodium sulfite is from about is from about 1:1.5 to about 1:4.5.

In some embodiments, the antifolate is pemetrexed and the weight ratio of cysteine hydrochloride to sodium sulfite is from about is from about 1:1.5 to about 1:4.5.

In some embodiments, the antifolate is pemetrexed and the antifolate is present in the pharmaceutical formulation at a concentration from about 0.5 mg/mL to about 50 mg/mL, or from about 10 mg/mL to about 40 mg/mL, or from about 20 mg/mL to about 30 mg/mL, or from about 22.5 mg/ml to about 27.5 mg/ml, for example about 25 mg/mL

In some embodiments, the osmolality of the pharmaceutical formulation is from about 250 mOsm to about 400 mOsm and the antifolate is present in the pharmaceutical formulation at a concentration from about 0.5 mg/mL to about 50 mg/mL, or from about 10 mg/mL to about 40 mg/mL, or from about 20 mg/mL to about 30 mg/mL, or from about 22.5 mg/ml to about 27.5 mg/ml, for example about 25 mg/mL

In some embodiments, the weight ratio of the antifolate to cysteine hydrochloride is from about 12:1 to about 55:1, or about 13:1 to about 25:1, or about 14:1 to about 18:1, and the weight ratio of the antifolate to sodium sulfite is from about 2.5:1 to about 22:1, or from about 1:1.5 to about 1:2.5, or from about 4:1 to about 9:1. The antifolate may be pemetrexed.

In some embodiments, the weight ratio of the antifolate to cysteine hydrochloride is from about 12:1 to about 55:1, or about 13:1 to about 25:1, or about 14:1 to about 18:1 and the weight ratio of cysteine hydrochloride to sodium sulfite is from about is from about 1:1.5 to about 1:4.5. The antifolate may be pemetrexed.

In some embodiments, the weight ratio of the antifolate to cysteine hydrochloride is from about 12:1 to about 55:1, or about 13:1 to about 25:1, or about 14:1 to about 18:1, and the weight ratio of the antifolate to sodium sulfite is from about 2.5:1 to about 22:1, or from about 1:1.5 to about 1:2.5, or from about 4:1 to about 9:1, and the weight ratio of cysteine hydrochloride to sodium sulfite is from about is from about 1:1.5 to about 1:4.5. The antifolate may be pemetrexed.

In some embodiments, the weight ratio of the antifolate to cysteine hydrochloride is from about 12:1 to about 55:1, or about 13:1 to about 25:1, or about 14:1 to about 18:1, and the weight ratio of the antifolate to sodium sulfite is from about 2.5:1 to about 22:1, or from about 1:1.5 to about 1:2.5, or from about 4:1 to about 9:1, and the weight ratio of cysteine hydrochloride to sodium sulfite is from about is from about 1:1.5 to about 1:4.5, or from about 1:2.8 to about 1:3.1 and the osmolality of the pharmaceutical formulation is from about 250 mOsm to about 400 mOsm.

In some embodiments, the weight ratio of the antifolate to cysteine hydrochloride is from about 12:1 to about 55:1, or about 13:1 to about 25:1, or about 14:1 to about 18:1, and the weight ratio of the antifolate to sodium sulfite is from about 2.5:1 to about 22:1, or from about 1:1.5 to about 1:2.5, or from about 4:1 to about 9:1, and the weight ratio of cysteine hydrochloride to sodium sulfite is from about is from about 1:1.5 to about 1:4.5.

In some embodiments, the antifolate is present in the aqueous solution in an amount of from about 2% w/w to about 10% w/w based on the total amount of aqueous solution, or about 2% w/w to about 8% w/w based on the total amount of aqueous solution, or from about 2.19% w/w to about 2.5% w/w or from about 4.0 to 5.5% w/w based on the total weight of the aqueous solution. In some embodiments, the cysteine hydrochloride monohydrate is present in the aqueous solution in an amount of from about 0.04% w/w to about 0.2% w/w based on the total amount of aqueous solution, or from 0.1% w/w to 0.2% w/w based on the total amount of aqueous solution. In some embodiments, the sodium sulfite is present in an amount of about 0.1% w/w to about 0.95% w/w based on the total amount of aqueous solution, or from 0.3% w/w to about 0.8% w/w based on the total amount of aqueous solution. In some embodiments, the aqueous solution comprises NaOH which is present in an amount from about 0.4 w/w % to about 0.9% w/w based on the total amount of the aqueous solution. In some embodiments, the aqueous solution comprises about 2% w/w to about 10% w/w of antifolate, 0.04% w/w to about 0.2% w/w cysteine hydrochloride monohydrate and 0.1% w/w to about 0.95% w/w sodium sulfite based on the total amount of aqueous solution and optionally 0.4% w/w to about 0.9% w/w NaOH based on the total amount of the aqueous solution. In some embodiments, the aqueous solution comprises 94% w/w to 97.9% w/w water based on the total amount of the aqueous solution. In preferred embodiments, the antifolate is pemetrexed. In some preferred embodiments, the osmolality of the pharmaceutical formulation is from about 250 mOsm to about 400 mOsm.

For the avoidance of doubt, the present application is directed to subject-matter described in the following numbered paragraphs.

1. A method of treating a viral pulmonary infection, the method comprising administering to a subject in need thereof a therapeutically effective amount of an antifolate.

2. The method according to numbered paragraph 1, wherein the antifolate or a metabolite thereof is a dihydrofolate reductase (DHFR) inhibitor, a thymidylate synthase (TS) inhibitor, and/or a glycinamide ribonucleotide transferase (GARFT) inhibitor.

3. The method according to numbered paragraph 2, wherein the antifolate metabolite is a mono- di- or poly-glutamated antifolate.

4. The method according to any one of numbered paragraphs 1 to 3, wherein the antifolate is a compound of formula (I), or a pharmaceutically acceptable salt thereof:

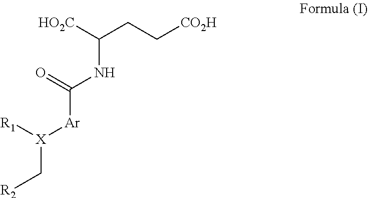

wherein Ar is selected from a 5-membered aromatic ring or 6-membered aromatic ring;
wherein X is selected from N or CH
wherein R₁ is selected from H, alkyl, alkenyl or alkynyl, and
wherein R₂ is selected from

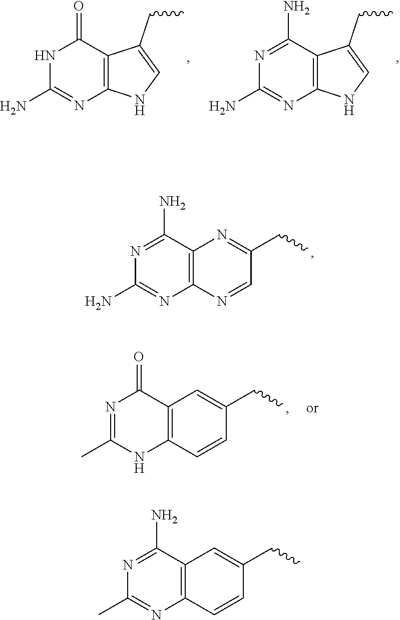

5. The method according to any of the preceding numbered paragraphs, wherein the antifolate is a compound of formula (II), or a pharmaceutically acceptable salt thereof

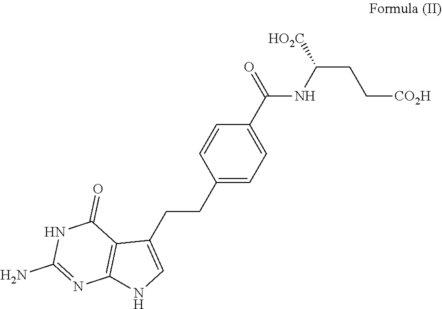 [SMILES: Nc1nc2[nH]cc(CCc3ccc(C(=O)N[C@@H](CCC(=O)O)C(=O)O)cc3)c2c(=O)[nH]1]

6. The method according to numbered paragraph 4 or numbered paragraph 5, wherein the compound of formula (I) or formula (II) is selected from the free acid or a salt, wherein the salt is selected from aluminum, arginine benzathine, chloroprocaine, calcium, choline, diethanolamine, ethanolamine, ethylenediamine, lysine, magnesium, lithium, histidine, sodium, potassium, tromethamine, meglumine, procaine, triethylamine or zinc.

7. The method according to any of the preceding numbered paragraphs, wherein the antifolate is not administered concurrently with folic acid or does not require pre-treatment with either folic acid or vitamin B12.

8. The method according to any of the preceding numbered paragraphs, wherein the viral pulmonary infection is a coronavirus infection, an influenza infection, or a respiratory syncytial viral infection.

9. The method according to any of the preceding numbered paragraphs, wherein the viral pulmonary infection is a coronavirus infection.

10. The method according to any of the preceding numbered paragraphs, wherein the viral pulmonary infection is COVID-19.

11. The method according to any of the preceding numbered paragraphs, wherein the antifolate is administered orally, parenterally, by inhalation or by nebulisation, or by intratracheal instillation.

12. The method according to any of the preceding numbered paragraphs, wherein the antifolate is administered parenterally, for example, to lung alveolar cells.

13. The method according to numbered paragraph 12, wherein the antifolate is administered parenterally by injection.

14. The method of numbered paragraph 13, wherein by injection is by intravenous injection, by intramuscular injection, by intradermal injection or by subcutaneous injection.

15. The method of numbered paragraph 13, wherein the antifolate is administered by intravenous bolus injection.

16. The method of numbered paragraph 15, wherein the antifolate is administered by intravenous bolus injection in multiple doses.

17. The method according to any of the preceding numbered paragraphs, wherein the antifolate is administered every 3 or 4 hours up to every 4 weeks.

18. The method according to any of the preceding numbered paragraphs, wherein the antifolate dosage is from about 0.01 mg/m² to about 700 mg/m² of body surface area, optionally from about 0.1 mg/m² to about 150 mg/m² of body surface area, further optionally from 25 mg/m² to 100 mg/m² of body surface area and even further optionally from about 35 mg/m² to about 75 mg/m² body surface area.

19. The method according to any of the preceding numbered paragraphs, wherein the amount of antifolate administered to the subject is from about 0.2 mg to about 300 mg, optionally from 1 to about 200 mg, optionally from 35 mg to about 175 mg, or further optionally from about 50 mg to about 150 mg.

20. The method according to any of the preceding numbered paragraphs, the method comprising administering to a subject in need thereof a therapeutically effective amount of an antifolate using a pharmaceutical formulation comprising the antifolate, cysteine hydrochloride and at least one of sodium metabisulfite, potassium metabisulfite, sodium bisulfite, or sodium sulfite.

21. The method according to any of the preceding numbered paragraphs, the method comprising administering to a subject in need thereof a therapeutically effective amount of an antifolate using a pharmaceutical formulation comprising the antifolate, sodium sulfite and cysteine hydrochloride.

22. A pharmaceutical formulation comprising an antifolate, sodium sulfite, and cysteine hydrochloride.

23. The pharmaceutical formulation according to numbered paragraph 22, wherein the antifolate or a metabolite thereof is a dihydrofolate reductase (DHFR) inhibitor, a thymidylate synthase (TS) inhibitor, and/or a glycinamide ribonucleotide transferase (GARFT) inhibitor.

24. The pharmaceutical formulation according to numbered paragraph 22 wherein the antifolate metabolite is a mono-, di-, or poly-glutamated antifolate.

25. The pharmaceutical formulation according to any one of numbered paragraphs 22 to 24, wherein the antifolate is a compound of formula (I), or a pharmaceutically acceptable salt thereof:

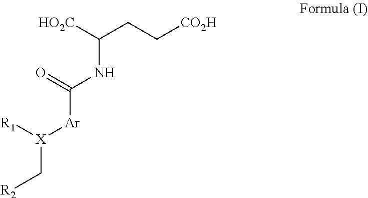

wherein Ar is selected from a 5-membered aromatic ring or 6-membered aromatic ring;
wherein X is selected from N or CH
wherein R₁ is selected from H, alkyl, alkenyl or alkynyl and
wherein R₂ is selected from

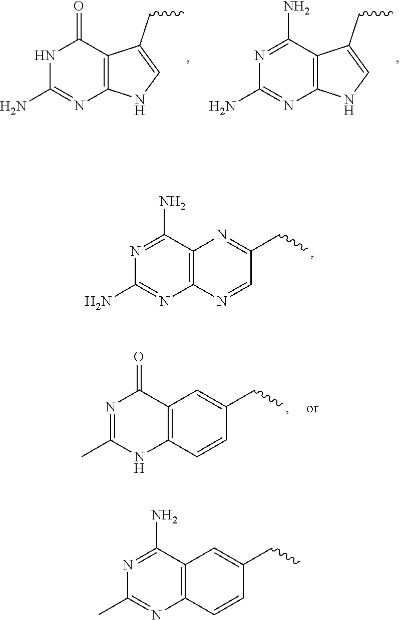

26. The pharmaceutical formulation according to any one of numbered paragraphs 22 to 25, wherein the antifolate is a compound of formula (II), or a pharmaceutically acceptable salt thereof:

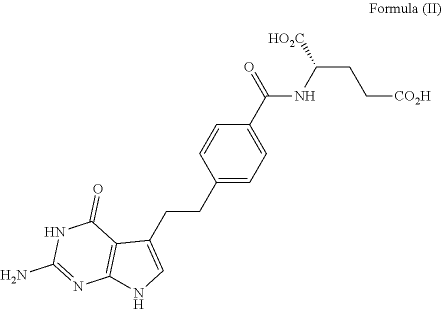 [SMILES: Nc1nc2[nH]cc(CCc3ccc(C(=O)N[C@@H](CCC(=O)O)C(=O)O)cc3)c2c(=O)[nH]1]

27. The pharmaceutical formulation according to numbered paragraph 25 or numbered paragraph 26, wherein the compound of formula (I) or formula (II) is selected from the free acid or a salt, wherein the salt form is selected from aluminum, arginine benzathine, chloroprocaine, calcium, choline, diethanolamine, ethanolamine, ethylenediamine, lysine, magnesium, lithium, histidine, sodium, potassium, tromethamine, meglumine, procaine, triethylamine or zinc.

28. The pharmaceutical formulation according to any one of numbered paragraphs 22 to 27, wherein the weight ratio of the antifolate to cysteine hydrochloride is from about 13:1 to about 25:1, or about 14:1 to about 18:1

29. The pharmaceutical formulation according to any one of numbered paragraphs 22 to 28, wherein the weight ratio of the antifolate to sodium sulfite is from about 1:1.5 to about 1:2.5, or about 4:1 to about 9:1.

30. The pharmaceutical formulation according to any one of numbered paragraphs 22 to 29, wherein the weight ratio of cysteine hydrochloride to sodium sulfite is from about 1:1.5 to about 1:4.5, or from about 1:2.8 to 1:3.1

31. The pharmaceutical formulation according to any one of numbered paragraphs 22 to 30, wherein the pharmaceutical formulation is in the form of an aqueous solution, a suspension, an emulsion or a powder.

32. The pharmaceutical formulation according to any one of numbered paragraphs 22 to 31, wherein the pharmaceutical formulation is in the form of an aqueous solution.

33. The pharmaceutical formulation according to numbered paragraph 32, wherein the aqueous solution has a pH from about 6.0 to about 7.5, or from about 6.5 to about 7.5.

34. The pharmaceutical formulation according to any one of numbered paragraphs 32 or 33, wherein the antifolate is present in the aqueous solution at a concentration from about 0.5 mg/mL to about 100 mg/mL in the aqueous solution, or from about 35 mg/mL to about 80 mg/mL.

35. The pharmaceutical formulation according to any one of numbered paragraphs 32 to 34, wherein the aqueous solution comprises about 2% w/w to about 10% w/w of antifolate, 0.04% w/w to about 0.2% w/w cysteine hydrochloride monohydrate and 0.1% w/w to about 0.95% w/w sodium sulfite based on the total amount of aqueous solution and optionally 0.4% w/w to about 0.9% w/w NaOH based on the total amount of the aqueous solution.

36. The pharmaceutical formulation according to any one of numbered paragraphs 32 to 35, wherein the aqueous solution is a ready-to-administer aqueous solution.

37. The pharmaceutical formulation according to any one of numbered paragraphs 32 to 36, wherein the aqueous solution is suitable for parenteral administration.

38. The pharmaceutical formulation according to any one of numbered paragraphs 32 to 37, wherein the aqueous solution is a diluted aqueous solution, diluted from a ready-to-dilute aqueous solution.

39. The pharmaceutical formulation according numbered paragraph 38, wherein the antifolate is present in the ready-to-dilute aqueous solution at a concentration of from about 1 mg/mL to about 100 mg/mL.

40. The pharmaceutical formulation according to numbered paragraph 38 or numbered paragraph 39, wherein the ready-to-dilute aqueous solution is diluted with a pharmaceutically acceptable solvent or intravenous fluid medium.

41. The pharmaceutical formulation according to numbered paragraph 40, wherein the aqueous solution is an aerosolized aqueous solution.

42. The pharmaceutical formulation according to numbered paragraph 41, wherein the aerosolized aqueous solution has a d90 particle size of less than about 10 μm, or less than about 5 μm.

43. The pharmaceutical formulation according to numbered paragraph 41 or numbered paragraph 42, wherein the aerosolized aqueous solution is suitable for inhalation or intratracheal instillation.

44. The pharmaceutical formulation according to any one of numbered paragraphs 22 to 43, further comprising one or more pharmaceutically acceptable excipients selected from chelating agents, amino acids, bulking agents, mucolytic agents, buffers, pH and tonicity adjusters, and combinations thereof.

45. The pharmaceutical formulation according to numbered paragraph 44, wherein the chelating agent is selected from EDTA and salts thereof, citric acid, malic acid, malonic acid, oxalic acid, succinic acid, tartaric acid or a combination thereof.

46. The pharmaceutical formulation according to numbered paragraph 44, wherein the amino acid is selected from histidine, glutamic acid, lysine, arginine, or a combination thereof.

47. The pharmaceutical formulation according to numbered paragraph 44, wherein the bulking agent is selected from dextrose, sucrose, mannose, mannitol or a combination thereof.

48. The pharmaceutical formulation according to numbered paragraph 44, wherein the mucolytic agent is N-acetyl cysteine.

49. The pharmaceutical formulation according to numbered paragraph 44, wherein the buffer is selected from hydrochloric acid, sodium hydroxide, tris, acetate, citrate, tartrate, phosphate, benzoate, bicarbonate, sodium chloride, potassium chloride or a combination thereof

50. The pharmaceutical formulation according to numbered paragraph 44, wherein the tonicity adjuster is selected from NaCl, KCl, glucose, dextrose, mannitol or glycerin.

51. The pharmaceutical formulation according to any one of numbered paragraphs 22 to 50, wherein the osmolality of the pharmaceutical formulation is from about 250 mOsm to about 400 mOsm.

### EXAMPLES

TABLE 1
Example Formulation Range for the pharmaceutical formulation

% (w/w)

| Ingredient | Low | High |
|---|---|---|
| Pemetrexed | 2.0 | 10.0 |
| Cysteine hydrochloride monohydrate | 0.04 | 0.2 |
| Sodium Sulfite | 0.1 | 0.95 |
| Sodium hydroxide | 0.4 | 0.9 |
| Water for injection | 94 | 97.9 |
| Optionally, NaOH or HCl acid for pH | | |
| adjustments | | |
| Optionally, additional NaCl or sugars (e.g. | | |
| glucose or dextrose) to adjust tonicity | | |

pH ranges from 6 to 7.5

Osmolality ranges between 270 to 350 mOsm

These formulations may be suitable for use as intravenous bolus or subcutaneous injection, without dilution.

Formulation Examples for the Antifolate Injection Solution

TABLE 2a

| | | Concentration | Material |
|---|---|---|---|
| Ingredient | % (w/w) | (mg/mL) | Grades |
| Pemetrexed Free acid | 2.5 | 25 | |
| Equivalent (from | | | |
| disodium salt form) | | | |
| Cysteine Hydrochloride | 0.17 | 1.72 | EP, USP |
| Monohydrate (on | | | |
| anhydrous basis) | | | |
| Sodium Sulfite, | 0.50 | 5.1 | NF, EP, JP, |
| Anhydrous | | | BP, FCC |
| Water for Injection | 96.86 | qs to 1 mL | USP, EP |
| Sodium hydroxide or | | | |
| hydrochloric acid | | | |
| solutions to adjust | | | |
| pH, as necessary | | | |

TABLE 2b

| | | Concentration | Material |
|---|---|---|---|
| Ingredient | % (w/w) | (mg/mL) | Grades |
| Pemetrexed Free acid | 5.0 | 50 | |
| Cysteine Hydrochloride | 0.16 | 1.60 | EP, USP |
| Monohydrate | | | |
| Sodium Sulfite, | 0.70 | 7.0 | NF, EP, JP, |
| Anhydrous | | | BP, FCC |
| Sodium Hydroxide | 0.96 | 9.6 | |
| Water for Injection | 93.18 | qs to 1 mL | USP, EP |
| Sodium hydroxide or | | | |
| hydrochloric acid | | | |
| solutions to adjust | | | |
| pH, as necessary | | | |

Example Manufacturing Process
1. Weigh raw materials
2. Add excipients to water for injection under inert gas atmosphere with stirring (should dissolve to a clear solution in <10 min) under ambient temperature
3. Add pemetrexed to solution (from step 2) and continue stirring under inert gas atmosphere (should dissolve in <10 min) under ambient temperature
4. Filter
5. Fill into suitable vial/container size (ideally under nitrogen, with head-space purged by nitrogen, as well)
6. Crimped close with aluminum flip-off cap
7. Sterilize by (moist) heat (steam-sterilization) at 121° C. for 12 to 30 min.

Moist heat sterilization (terminal steam-sterilization at 121° C. for ca. 15 min) results only in a loss of <1% of the potency (assayed concentration) of the drug.

Physical Properties of an Example Pharmaceutical Formulation and an Example Diluted Ready-to-Administer Aqueous Solution for IV Infusion

TABLE 3

| | | | | Following Dilution (×5) in |
|---|---|---|---|---|
| | Product Characteristics (e.g. | | | 0.9% NaCl Solution of the |
| | for direct IV bolus injection | | | 25 mg/mL solution (e.g. for use |
| | or subcutaneous injection, for | | | in IV infusion, for example, |
| | example, for anti-viral therapy) | | | for lung cancer treatment) |
| Pemetrexed | 75 mg/mL | 25 mg/mL | | 5 mg/mL |
| Concentration | | | | |
| pH | 6.5 | 7.2 | | 7.25 |
| Density | 1.0112 ± 0.01 g/mL² | 1.013 ± 0.001 g/mL¹ | | 1.006 g/mL² |
| Osmolality | 346 ± 10 mOsm | 292 ± 20 mOsm | | 271 ± 1 mOsm |
| Nominal Fill | 1 mL (for 75 mg) | 2 mL | (for 50 mg) | |
| Volumes³ | 2 mL (for 150 mg) | 4 mL | (for 100 mg) | |
| | | 20 mL | (for 500 mg) | |

| | | | |
|---|---|---|---|
| Viscosity | 1 cP | 1 cP | 1 cP |

¹measured at 22° C.
²measured at 24° C.
³Adjusted excess to allow for withdrawal of sufficient volume for dose

Stability Studies

The pharmaceutical formulation was tested for stability. An assay of the active and related substances is based a reversed phase HPLC method. The pharmaceutical formulation was found to be stable following steam-sterilization and at least 6 month storage at elevated temperatures (40° C.), as shown in Table 4 below.

TABLE 4
Assay and Area (%) of Related Substances (RS) listed
by Relative Retention Times (RRT) following sterilization
and storage for about 6 months at 40° C.

| | Condition | | | |
|---|---|---|---|---|
| | | | | 168 d at |
| | | Time 0 | Sterilized⁶ | 40° C. |
| | Assay (%) | | | |
| | | 100.0 | 99.20 | 101.6 |
| Related Substance | RRT | Related Substances | | |
| | | (% peak area of active) | | |
| Unknown | 0.20 | 0.05 | ND | 0.05 |
| Unknown | 0.22 | ND | ND | 0.06 |
| Unknown | 0.25 | 0.09 | 0.16 | ND |
| Epoxy-hemiaminal | 0.31 | ND | ND | 0.46 |
| Unknown | 0.40 | ND | 0.05 | ND |
| Unknown | 0.44 | 0.06 | 0.05 | ND |
| Unknown | 0.46 | 0.05 | 0.09 | 0.31 |
| Alpha-hydroxy lactam isomer 1 | 0.51 | ND | ND | 0.06 |
| Alpha-hydroxy lactam isomer 2 | 0.53 | ND | ND | 0.04 |
| Unknown | 0.59 | ND | 0.06 | ND |
| Unknown | 0.60 | ND | 0.12 | ND |
| Lactam isomer 1 | 0.66 | ND | ND | 0.07 |
| Lactam isomer 2 | 0.66⁷ | ND | ND | 0.13 |
| Unknown | 0.85 | ND | ND | 0.10 |
| Ring-opened keto-amine | 0.97 | ND | 0.10 | 0.09 |
| Unknown | 1.13 | 0.04 | ND | 0.04 |
| | Total | 0.29 | 0.63 | 1.39 |

⁶sample for steam-sterilized for 20 min at 121° C.
⁷Two different RS peaks at approximate RRT 0.66, with resolution < 1 (R < 1)

The above results demonstrate that the pharmaceutical formulations described herein have good stability. The stability at the laboratory scale listed above is expected to improve further for products that are manufactured and packaged under suitable controls implemented in a Good Manufacturing Practices (GMP) environment.

Anti-Viral Assay

For the following Anti-Viral Assay and Results, Emphascience, Inc. has utilized the non-clinical and pre-clinical services program offered by the National Institute of Allergy and Infectious Diseases.

Confluent or near-confluent cell culture monolayers of the tested cell-type are prepared in 96-well disposable microplates the day before testing. Cells are maintained in MEM supplemented with 2% FBS supplemented with 50-μg/ml gentamicin. The tested sample/compound is diluted with water for the preparation of eight half-log₁₀ concentrations over the range of 0.1 and 100 μg/ml or μM. Five microwells are used per dilution: three for infected cultures and two for uninfected toxicity cultures. Controls for the experiment consist of six microwells that are infected and not treated (virus controls) and six that are untreated and uninfected (cell controls) on every plate. A known active drug (M128533) is tested in parallel as a positive control drug using the same method as is applied for test compounds. The positive control is tested with every test run. After sufficient virus replication occurs (3 days for SARS-CoV-2), a sample of supernatant is taken from each infected well (three replicate wells are pooled) and tested immediately or held frozen at −80° C. for later virus titer determination. After maximum CPE is observed, the viable plates are stained with neutral red dye. The incorporated dye content is quantified as described above to generate the EC₅₀ and CO₅₀ values.

The VYR test is a direct determination of how much the test compound inhibits virus replication. Virus yielded in the presence of test compound is titrated and compared to virus titers from the untreated virus controls. Titration of the viral samples (collected as described in the paragraph above) is performed by endpoint dilution as is known in the art (e.g. the Reed-Reed, L. J., and H. Muench. “A Simple Method of Estimating Fifty Percent Endpoints). Serial 1/10 dilutions of virus are made and plated into 4 replicate wells containing fresh cell monolayers of Caco-2 cells. Plates are then incubated, and cells are scored for presence or absence of virus after distinct CPE is observed, and the CCID₅₀ calculated using the Reed-Muench method). The 90% (one log₁₀) effective concentration (EC₉₀) is calculated by regression analysis by plotting the log₁₀ of the inhibitor concentration versus log₁₀ of virus produced at each concentration. Dividing EC₉₀ by the CO₅₀ gives the Selectivity Index-90 (SI₉₀) value for this test

TABLE 5

| | |
|---|---|
| Virus Screened | SARS-CoV-2 |
| Virus Strain | USA_WA1/2020 |
| Cell line | Caco-2¹ |
| Vehicle | Water |
| Drug | Pemetrexed Formulation (EMP-201) |
| Drug Conc. Range | 0.032-100 μg/ml |
| Control | M128533 |
| Control Conc. Range | 0.032-100 μg/ml |

¹Caco-2 cells have been extensively used to study infection with SARS-CoV and are an established model for SARS-CoV-2 infection

TABLE 6
Results: Visual Virus Yield Reduction
and Control Red Toxicity Assay

| Drug | EC₉₀ | CC₅₀ | SI₉₀ |
|---|---|---|---|
| M128533 | 4.6 | >100 | 22 |
| EMP-201 | 4.5 | 94 | 21 |
| (added at time | | | |
| of infection) | | | |
| EMP-201 | 1.4 | 74 | 53 |
| (added 2 days | | | |
| post-infection) | | | |

EMP-201 corresponds to a pemetrexed formulation comprising sodium sulfite and cysteine hydrochloride monohydrate as indicated in the Example formulations above
EC₉₀—compound concentration that reduces viral replication by 90%
CC₅₀—compound concentration that reduces cell viability by 50%
Selectivity Index for 90% Inhibition of Viral Replication (SI₉₀) = CC₅₀/EC₉₀

The results above show that pemetrexed has similar or better anti-viral properties against SARS-CoV-2, as compared to the positive control M128533. Since the ratio of CC₅₀ to EC₉₀ is >10, the results also demonstrate that treatment with pemetrexed would likely have an acceptable safety window making it a viable candidate for in-vivo testing in diseased animal models. The results also demonstrate that such activity against SARS-CoV-2 is even better (lower EC₉₀ and higher CO₅₀, when pemetrexed is added to the cell culture two days after the cells had been infected with the SARS-CoV-2 virus. Such improved activity well into the infection could offer a much-needed therapeutic option against viral pulmonary infections such as SARS-CoV-2, when treatment and intervention is delayed and a patient is already experiencing acute manifestations of the disease.

Dosing

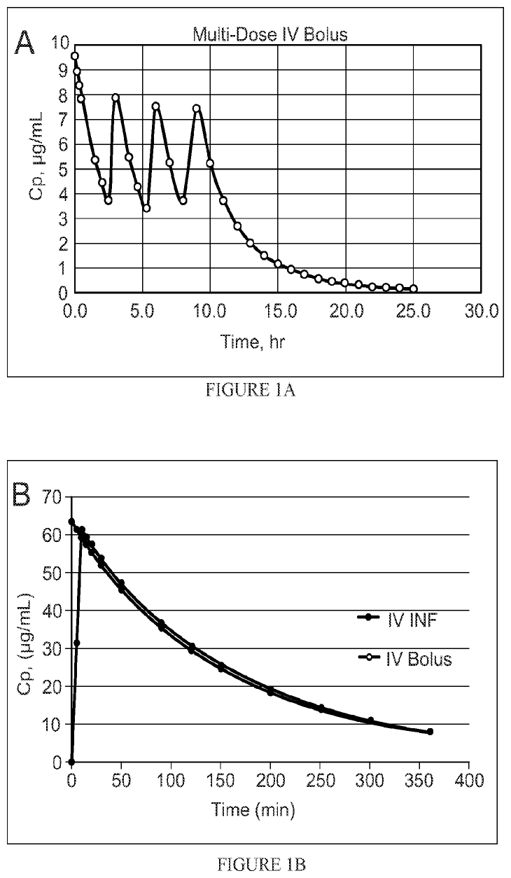

FIG. 1 shows the simulated plasma profile from 4 doses of IV bolus injections of the drug administered 3 hr apart starting with a dose of 150 mg, and followed by 3×75 mg doses compared to the plasma profile from one 1000 mg dose (either bolus or 10 min IV infusion) as currently used in cancer chemotherapy. The pharmacokinetic parameters for this simulation are from population pharmacokinetic parameters of the currently marketed reference listed drug product containing pemetrexed (Alimta®). This method of administering frequent and multiple smaller doses by IV bolus injection can be used to maintain the plasma concentration at around 4 μg/mL at or near the EC90 in Caco-2 cells infected with SARS-CoV-2 for >10 hr while limiting the peak plasma concentrations to be at least 7× lower than the CC50 measured by VYR assay (≥74 μg/mL) in the same cell line. The total administered dose with such an approach would be 375 mg in a single day and less than the 500 mg/m² (or roughly 1000 mg) given to patients for cancer chemotherapy. An example product in accordance with this invention with a concentration of 75 mg/mL, at a pH 6.5 and an osmolality of 346 mOsm provides a formulation that is convenient for administering bolus IV injections every 3 hrs. This effectively extends the plasma levels without using excessively high doses.

1. A method of treating a viral pulmonary infection, the method comprising administering to a subject in need thereof a therapeutically effective amount of an antifolate.

2. The method according to claim 1, wherein the antifolate is a compound of formula (I), or a pharmaceutically acceptable salt thereof: 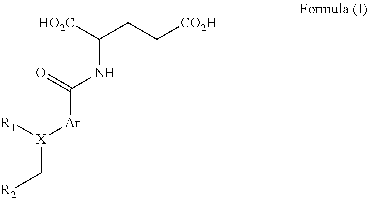
wherein Ar is selected from a 5-membered aromatic ring or 6-membered aromatic ring;
wherein X is selected from N or CH
wherein R₁ is selected from H, alkyl, alkenyl or alkynyl, and
wherein R₂ is selected from

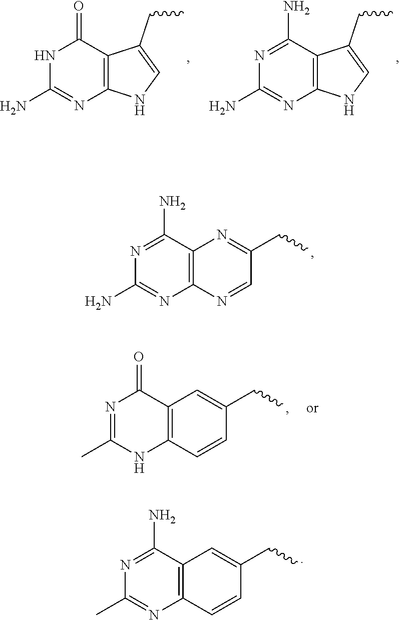

3. The method according to claim 1, wherein the antifolate is a compound of formula (II), or a pharmaceutically acceptable salt thereof

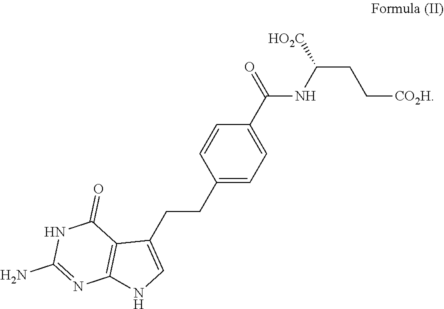 [SMILES: Nc1nc2[nH]cc(CCc3ccc(C(=O)N[C@@H](CCC(=O)O)C(=O)O)cc3)c2c(=O)[nH]1]

4. The method according to claim 2, wherein the compound of formula (I) or formula (II) is selected from the free acid or a salt, wherein the salt is selected from aluminum, arginine benzathine, chloroprocaine, calcium, choline, diethanolamine, ethanolamine, ethylenediamine, lysine, magnesium, lithium, histidine, sodium, potassium, tromethamine, meglumine, procaine, triethylamine or zinc.

5. The method according to claim 1, wherein the viral pulmonary infection is a coronavirus infection, an influenza infection, or a respiratory syncytial viral infection.

6. The method according to claim 1, wherein the viral pulmonary infection is COVID-19.

7. The method according to claim 1, wherein the antifolate is administered parenterally by injection.

8. The method of claim 7, wherein the antifolate is administered parenterally by intravenous injection, by intramuscular injection, by intradermal injection or by subcutaneous injection.

9. The method according to claim 1, wherein the antifolate dosage is from about 0.01 mg/m² to about 700 mg/m² of body surface area.

10. The method according to claim 1, wherein the amount of antifolate administered to the subject is from about 0.2 mg to about 300 mg.

11. The method according to claim 1, the method comprising administering to a subject in need thereof a therapeutically effective amount of an antifolate using a pharmaceutical formulation comprising the antifolate, cysteine hydrochloride and at least one of sodium metabisulfite, potassium metabisulfite, sodium bisulfate, or sodium sulfite.

12. The method according to claim 1, the method comprising administering to a subject in need thereof a therapeutically effective amount of an antifolate using a pharmaceutical formulation comprising the antifolate, sodium sulfite and cysteine hydrochloride.

13. The method according to claim 11, wherein the pharmaceutical formulation is in the form of an aqueous solution.

14. The method according to claim 13, wherein the aqueous solution comprises about 2% w/w to about 10% w/w of antifolate, 0.04% w/w to about 0.2% w/w cysteine hydrochloride monohydrate and 0.1% w/w to about 0.95% w/w sodium sulfite based on the total amount of aqueous solution and optionally 0.4% w/w to about 0.9% w/w NaOH based on the total amount of the aqueous solution.

15. The method according to claim 1, wherein the osmolality of the pharmaceutical formulation is from about 250 mOsm to about 400 mOsm.

16. The method according to claim 8, wherein the antifolate is administered by intravenous bolus injection.

17. The method according to claim 8, wherein the antifolate is administered by intravenous bolus injection in multiple doses.

18. The method according to claim 9, wherein the antifolate dosage is from about 0.1 mg/m² to about 150 mg/m² of body surface area.

19. The method according to claim 9, wherein the antifolate dosage is from about 25 mg/m² to about 100 mg/m² of body surface area.

20. The method according to claim 9, wherein the antifolate dosage is from about 35 mg/m² to about 75 mg/m² of body surface area.
